(19) United States
(12) Patent Application Publication
(10) Pub. No.: US 2026/0020891 A1
(43) Pub. Date: Jan. 22, 2026

(54) METHODS AND SYSTEMS FOR DENSE SPEED OF SOUND SHIFT IMAGING

(71) Applicant: RAMOT AT TEL AVIV UNIVERSITY LTD., Tel Aviv (IL)

(72) Inventors: Tali ILOVITSH, Tel Aviv (IL); Tal GRUTMAN, Tel Aviv (IL); Keren KARLINSKY, Tel Aviv (IL)

(21) Appl. No.: 19/346,686

(22) Filed: Oct. 1, 2025

Related U.S. Application Data
(63) Continuation of application No. PCT/IL2024/050360, filed on Apr. 11, 2024.
(60) Provisional application No. 63/459,216, filed on Apr. 13, 2023.

(51) Int. Cl. A61B 18/04; A61B 8/08; A61B 18/00; G01K 11/24

(52) U.S. Cl. CPC A61B 18/04; A61B 8/08; G01K 11/24; A61B 2018/00041; A61B 2018/00577; A61B 2018/00642; A61B 2018/00803

(57) ABSTRACT

Disclosed are methods and systems, comprising real-time dense algorithms calculating the speed-of-sound shift between acoustic acquisitions, that allows enhanced noninvasive temperature evaluation during treatment such as thermal ablation.

### CROSS REFERENCE TO RELATED APPLICATIONS

This application is a Bypass Continuation of PCT Patent Application No. PCT/IL2024/050360 having International filing date of Apr. 11, 2024, which claims the benefit of priority of U.S. Provisional Patent Application No. 63/459,216, filed Apr. 13, 2023, the contents of which are all incorporated herein by reference in their entirety.

### TECHNICAL FIELD

The present disclosure generally relates to ultrasound (US)-based monitoring of the internal sound speed change of tissue undergoing physiological change, such as during thermal ablation and cryo-ablation, using dense speed-of-sound shift imaging (DSI).

### BACKGROUND OF THE INVENTION

Ultrasound is a safe, cheap, and portable imaging modality with excellent temporal characteristics. In today's world, the development of new ultrasound-based methods for clinical guidance is motivated by the quality and availability of ultrasound transducers.

One ultrasound domain that has taken longer to integrate into the clinic is high-intensity focused ultrasound (HIFU). In this application, a sound wave is focused into tissue to release high energy and thus initiate heating beneath the skin surface. HIFU therapy is considered effective for various uses ranging originally from lesion destruction in the central nervous system to treatment of a wide array of tumors and cancers including uterine fibroids, prostate cancer, breast cancer, liver cancer, and more. Tumor necrosis is induced by the HIFU beam as the surrounding tissue is heated to temperatures at which the local damage is irreversible.

HIFU is a practical, non-invasive surgery. However, HIFU suffers from one clear drawback: no feedback is available regarding the status of the ongoing surgery. As energy is released into the tissue and the local thermal dose rises, it is crucial to monitor the focal area to verify that the temperature in the surrounding healthy tissue is maintained at a safe operating range and unintentional permanent damage is not induced.

The current standard for monitoring of temperature during HIFU surgeries utilizes MRI, producing complex systems where ultrasound transducers are tailor made to operate within the magnetic field of an MR scanner. Magnetic resonance-guided high-intensity focused ultrasound (MRgHIFU) has shown promising capabilities for measuring temperatures. In particular, the Proton Resonance Frequency (PRF) shift method, which utilizes the hydrogen bonds in water molecules to measure temperature change. However, PRF shift is notably affected by respiratory motion and is unable to detect temperature change in fat and bone, where no water molecules are present.

Other MR guided procedures have been invented to address this, most notably Acoustic Radiation Force Imaging (MR-ARFI), which encodes motion into the phase of the MR image, capturing the expected tissue motion due to respiration alongside displacement of tissue due to the HIFU treatment. Practical MR-ARFI techniques are still being invented to improve the temporal resolution, but the high cost of MRI is still a limiting factor in such setups.

Thus, although magnetic resonance imaging (MRI) is the gold standard for real-time temperature monitoring, its limited availability and high cost impede widespread usage of HIFU therapy (i.e., thermal ablation).

Perhaps the most cost-effective solution for HIFU guidance is thermometry acquired by a second ultrasound transducer, which offers a cheaper setup with potential temporal resolution at several orders of magnitude higher than MRI. Ultrasound setups using coherent plane wave compounding have achieved frame rates above 1 kHz, and usually settle on a signal-to-noise ratio/frame rate tradeoff that yields 50-500 frames per second. The basic principle of MR-ARFI is to measure tissue pixel displacement resulting from HIFU, which is not necessarily specific to MRI.

HIFU inflicted necrosis inherently changes the speed of sound in tissue, causing a measurable latency in the echo over successive ultrasound frames captured by an imaging transducer. This was originally measured using transmission-mode ultrasound measuring the time-of-flight, which is largely impractical in the clinic. Several works introduced a simpler method relating the measured echo shift between B-Mode images directly to the induced temperature change. This approach, later named thermal strain imaging (TSI), serves as the basis for many of the more recent developments in the field of ultrasonic thermometry. Thermal strain is impartial to the underlying ultrasound acquisition method or heating procedure. Thermal strain was measured using Pulse-Inversion Harmonic Imaging (PIHI), and a similar result was achieved using coherent plane wave compounding. Classical synthetic aperture setup with motion compensation was also used to show feasibility for a full ultrasound guided HIFU (USgHIFU) procedure. A similar approach to thermal strain, named Change in Backscattered Energy (CBE) has also been used for USgHIFU.

The main challenge for thermal strain imaging is measuring the echo shift between two successive ultrasound images. Several of the methods mentioned previously rely on cross-correlation to predict the echo shift, while others used Loupas' Estimator but these are computationally burdensome calculations for a method that is supposed to improve temporal resolution. Additionally, phase aliasing artifacts common to typical ultrasound imaging methods must be filtered after the echo shift is calculated, often incorporating heavy image processing including filters such as Savitzky-Golay.

Therefore, although thermal strain imaging (TSI) can provide ultrasound-guided temperature estimation, current TSI algorithms lack spatial coherence, and importantly require extensive computation, which reflects on the speed of the calculations, and the overall time it takes to calculate the temperature change, and therefore limits their effectiveness in providing accurate real-time monitoring of physiological changes during treatment.

Thus, there is a need for accurate, and fast, ultrasound-based thermometry measurements, monitoring the internal tissue temperature, for achieving effective thermal ablation and cryo-ablation, and preventing unwanted damage to surrounding tissues.

### SUMMARY OF THE INVENTION

According to some aspects, the present disclosure provides methods and systems, including computer-implemented methods, for real-time non-invasive monitoring of a change in internal speed-of-sound in biological tissue, and which can also be used for monitoring changes in temperature of the tissue, indicative of a treatment applied on the tissue in vivo or ex vivo.

It is important to consistently supervise the treatment during the application thereof on the biological tissue in order to avoid unwanted damage since the treatment can affect the temperature of the tissue, and can even destroy the tissue, for example, by inducing necrosis, cavitation and/or heat shock to the cells.

Advantageously, the methods and systems provided here include a Dense Sped-of-sound Shift Imaging (DSI) algorithm, configured to calculate a change in the internal sound speed (m/μsec) of the biological tissue based on a pixel shift (μm) determined between two successive ultrasound images. The DSI algorithm calculates the change in the internal sound speed of the biological tissue as a solution for an inverse problem.

Advantageously and surprisingly, in some embodiments, solving the inverse problem, presented in equation 3 provided herein elsewhere in the theoretical part, is a simpler and faster approach than solving state-of-the-art equation 1, also provided herein elsewhere in the theoretical part, therefore, the resulted calculation of a change in the internal sound speed includes an unexpected and surprisingly fast average processing speed of about 3.0 frames per second, which represents about 20-fold increase in speed with respect to using a state-of-the-art Thermal Strain Imaging for calculating a corresponding strain.

Further advantageous is the DSI algorithm being devoid of image processing/filtering. According to some embodiments, this facilitates at least some of the increase in the processing speed/the calculation time of the change in internal sound speed.

The calculated change in the internal sound speed (m/μsec) of the biological tissue may then be translated to a corresponding change in temperature by a simple act of computational conversion of units using a pre-determined factor.

Importantly, the DSI provides accurate temperature assessment based on the calculated change in speed-of-sound, with low variance, and in addition to its fast calculation time the herein disclosed methods and systems that utilize the DSI algorithm provide an effective and advantageous means of monitoring treatments that can affect the speed sound and/or temperature of the tissue, thereby, in some embodiments, reducing or preventing unwanted damage to the treated area of the tissue and its surrounding.

Even more, in some embodiments, the reconstructed change in the internal sound speed (m/μsec) of the biological tissue includes improved spatial coherence relative a reconstructed strain (%) using state-of-the-art TSI. Consequently, the DSI algorithm is characterized by determining the size of a focal area (mm) of a lesion as having a reduced size relative to a size of a focal area (mm) of a same lesion determined by Thermal Strain Imaging (TSI).

According to one aspect, the method for non-invasive monitoring of a change in internal sound speed of a biological tissue indicative of a treatment, comprises the steps of:
(a) acquiring at least two successive ultrasound (US) images of a target area of a biological tissue, thereby obtaining raw radio frequency (RF) data of the acquired US images; and
(b) applying onto the obtained data a Dense Speed-of-Sound Shift Imaging (DSI) algorithm configured to:
   (i) determine pixel shift (μm) between the at least two successive ultrasound images;
   (ii) calculate a change in the internal sound speed (m/μsec) of the biological tissue based on the pixel shift; and
wherein the DSI algorithm is characterized by solving an inverse problem; and wherein the calculated change in the internal sound speed of the biological tissue comprises the solution to the inverse problem.

According to one embodiment, the method estimates internal temperature change of the biological tissue based on the calculated change in the internal sound speed of the biological tissue.

According to some embodiments, the acquisition of at least two successive ultrasound (US) images of a target area of a biological tissue is performed during a treatment performed on the tissue thereby providing indication about the treatment; and wherein the treatment can inflict a physiological change on the inherent speed-of-sound of the biological tissue and/or wherein the treatment can inflict a physiological change in the internal temperature of the biological tissue. Each possibility is a separate embodiment.

According to additional embodiments, the method comprises a step of feeding back on the treatment; the feedback comprises providing a user with the calculated change in the inherent speed-of-sound of the biological tissue and/or with the calculated change in the internal temperature of the biological tissue. Each possibility is a separate embodiment.

According to further embodiments, the method comprises a step of feeding back on the treatment; the feedback comprises providing a user with a recommendation(s) at least about terminating or continuing the treatment and/or about adjusting treatment parameters. Each possibility is a separate embodiment.

According to other embodiments, the method comprises iterations of at least steps (a)-(b) of the method thereby allowing real-time non-invasive monitoring of a change in the internal sound speed of the biological tissue and/or a change in the internal temperature of the biological tissue. Each possibility is a separate embodiment.

In one embodiment, the acquisition of at least two successive ultrasound (US) images comprises acquiring plane waves B-mode data.

In another embodiment, the acquisition of at least two successive ultrasound (US) images comprises acquiring at least nine plane waves per image.

In specific embodiments, the at least nine plane waves per image comprises at least three plane angles having intervals selected from a range of intervals between about 5° and about 35°; and wherein each plane angle further comprises at least three angles having intervals selected from a range of intervals between about 0.1° and about 1.5°. Each possibility is a separate embodiment.

In one embodiment, the calculation of the change in internal sound speed (m/μsec) comprises processing speed of at least about 0.3 frames per second.

In a related embodiment, the calculation of the change in internal sound speed (m/μsec) comprises processing speed of at least about 1.0 frames per second.

In another related embodiment, the calculation of the change in internal sound speed (m/μsec) comprises processing speed of at least about 2.0 frames per second.

In further related embodiment, the calculation of the change in internal sound speed comprises DSI algorithm processing speed between about 0.3 frames per second and about 3.0 frames per second, corresponding to increased speed of between about 2-fold and about 21-fold relative to a processing speed (frame per second) of a corresponding calculation of a change in strain (%) using Thermal Strain Imaging (TSI).

In some embodiments, the calculation of a change in the internal sound speed of the biological tissue comprises determining a size of a focal area of a lesion; and wherein the DSI algorithm determines the size of a focal area (mm) of a lesion as having a reduced size relative to a size of a focal area (mm) of a same lesion determined by Thermal Strain Imaging (TSI); wherein the reduction includes reduction in FWHM in the lateral direction.

In some embodiments, the calculation of the change in internal sound speed is devoid of image processing/filtering; thereby allowing increasing processing speed of the calculation time of the change in internal sound speed.

According to some embodiments, the treatment comprises mechanical procedure, non-mechanical procedure and/or non-invasive procedure, or any combination thereof. Each possibility is a separate embodiment.

According to related embodiments, the treatment comprises one or more selected from: high-intensity focused ultrasound (HIFU) thermal ablation therapy, cryo-ablation, adhesive bonding assessment, mechanical ablation, histotripsy, introducing a drug or other biological or chemical compound into the tissue, contrast agents, and polymer congealing, or any combination thereof; and wherein said treatment can inflict a physiological change on the inherent speed-of-sound and/or the treatment can inflict a physiological change in the internal temperature of the biological tissue. Each possibility is a separate embodiment.

According to specific embodiments, the treatment comprises one or more selected from: high-intensity focused ultrasound (HIFU) thermal ablation therapy, cryo-ablation, and contrast agents, or any combination thereof. Each possibility is a separate embodiment.

In one embodiment, the acquiring of the at least two successive ultrasound (US) images is performed from a separate transducer than the HIFU transducer.

In another embodiment, the thermal ablation therapy comprises tumor ablation.

In another embodiment, the thermal ablation therapy comprises contrast agents.

In specific embodiments, the contrast agent is selected from one or more of nanobubbles, nanodroplets, microbubbles, and gas vesicles, or any combination thereof. Each possibility is a separate embodiment.

In some embodiments, the biological tissue comprises in-vivo biological tissue and/or ex-vivo biological tissue. Each possibility is a separate embodiment. In some embodiments, the biological tissue comprises a tumor.

In some embodiments, the biological tissue comprises one or more tissue selected from a soft tissue, solid tissue, fat, bone, cartilage, or chemical deposit, lymphatic vessel or a blood vessel, or any combination thereof. Each possibility is a separate embodiment.

According to another aspect, the system for real-time non-invasive monitoring of a change in internal sound speed of a biological tissue, comprises:
(a) at least one transducer capable of acquiring at least two successive ultrasound (US) images of a target area of a biological tissue;
(b) a transmitter capable of beamforming obtained raw radio frequency (RF) data of the acquired US images;
(c) a processing unit capable of receiving the RF data and executing thereon a Dense Speed-of-Sound Shift Imaging (DSI) algorithm configured to:
   (i) determine pixel shift (μm) between the at least two successive ultrasound images; and
   (ii) calculate a change in the internal sound speed (m/μsec) of the biological tissue based on the pixel shift; and
(d) an output device capable of presenting the calculated change in the sound speed; and
wherein the DSI algorithm is characterized by solving an inverse problem; and wherein the calculated change in the internal sound speed of the biological tissue comprises the solution to the inverse problem comprises.

According to one embodiment, the processing unit is configured to estimate internal temperature change of the biological tissue based on the calculated change in the internal sound speed of the biological tissue; and provide a presentation of the calculated change in temperature on the output device.

According to some embodiments, the acquisition of at least two successive ultrasound (US) images of a target area of a biological tissue is performed during a treatment performed on the tissue thereby providing indication about the treatment; and wherein the treatment can inflict a physiological change on the inherent speed-of-sound of the biological tissue and/or wherein the treatment can inflict a physiological change in the internal temperature of the biological tissue. Each possibility is a separate embodiment.

According to additional embodiments, the processing unit is configured to feedback on the treatment; the feedback comprises providing a user with a recommendation(s) at least about terminating or continuing the treatment and/or about adjusting treatment parameters; and wherein said recommendation(s) are presented to the user by same or different output device. Each possibility is a separate embodiment.

In some embodiments, the transducer comprises a power output unit with at least one characteristic selected from: a duty cycle of about 50%, frequency of about 2 MHz, and PRF of about 20 μsec, or any combination thereof. Each possibility is a separate embodiment.

In some embodiments, the transducer is capable of a plane steering acquisition control allowing acquisition of at least nine plane waves per image.

In specific embodiments, the at least nine plane waves per image comprises at least three plane angles having intervals selected from a range of intervals between about 5° and about 35°; and wherein each plane angle further comprises at least three angles compounded together having intervals selected from a range of intervals between about 0.1° and about 1.5°. Each possibility is a separate embodiment.

In one embodiment, the calculation of the change in internal sound speed (m/μsec) comprises processing speed of at least about 0.3 frames per second.

In a related embodiment, the calculation of the change in internal sound speed (m/μsec) comprises processing speed of at least about 1.0 frames per second.

In another related embodiment, the calculation of the change in internal sound speed (m/μsec) comprises processing speed of at least about 2.0 frames per second.

In further related embodiment, the calculation of the change in internal sound speed comprises DSI algorithm processing speed between about 0.3 frames per second and about 3.0 frames per second, corresponding to increased speed of between about 2-fold and about 21-fold relative to a processing speed (frame per second) of a corresponding calculation of a change in strain (%) using Thermal Strain Imaging (TSI).

In some embodiments, the calculation of the change in internal sound speed is devoid of image processing/filtering; thereby allowing increasing processing speed of the calculation time of the change in internal sound speed.

According to some embodiments, the treatment comprises one or more selected from: high-intensity focused ultrasound (HIFU) thermal ablation therapy, cryo-ablation, adhesive bonding assessment, mechanical ablation, histotripsy, introducing a drug or other biological or chemical compound into the tissue, contrast agents, and polymer congealing, or any combination thereof; and wherein said treatment can inflict a physiological change on the inherent speed-of-sound or the internal temperature of the biological tissue. Each possibility is a separate embodiment.

In one embodiment, the acquisition of at least two successive ultrasound (US) images is performed from a separate transducer than the HIFU transducer.

According to yet another aspect, the computer-implemented method for calculating a change in internal sound speed of a biological tissue indicative of a treatment, is configured to:
(a) receiving raw radio frequency (RF) data of at least two successively acquired ultrasound (US) images of a target area of a biological tissue;
(b) applying onto the data a Dense Speed-of-Sound Shift Imaging (DSI) algorithm that:
   (i) measures echo/pixel shift (μm) between the at least two successive ultrasound images; and
   (ii) calculates a change in internal sound speed (m/μsec) of the biological tissue based on the echo/pixel shift; and
(c) providing an output comprising the calculated change in the internal sound speed of the biological tissue; and
wherein the DSI algorithm is characterized by solving an inverse problem; and wherein the calculated change in the internal sound speed of the biological tissue comprises the solution to the inverse problem comprises.

According to one embodiment, the computer-implemented method estimates internal temperature change of the biological tissue based on the calculated change in the internal sound speed of the biological tissue; and provide an output comprising the calculated change in temperature.

According to some embodiments, the acquisition of at least two successive ultrasound (US) images of a target area of a biological tissue is performed during a treatment performed on the tissue; thereby providing indication about the treatment; and wherein the treatment can inflict a physiological change on the inherent speed-of-sound of the biological tissue and/or wherein the treatment can inflict a physiological change in the internal temperature of the biological tissue. Each possibility is a separate embodiment.

According to additional embodiments, the computer-implemented method is configured to provide an output comprising the calculated change in the internal temperature of the biological tissue.

According to further embodiments, the computer-implemented method is configured to provide an output comprising feedback on the treatment; the feedback comprises a recommendation(s) at least about terminating or continuing the treatment and/or about adjusting treatment parameters. Each possibility is a separate embodiment.

According to other embodiments, the computer-implemented method comprises iterations of at least steps (a)-(c) thereby allowing real-time non-invasive monitoring of a change in the internal sound speed of the biological tissue and/or a change in the internal temperature of the biological tissue. Each possibility is a separate embodiment.

In one embodiment, the calculation of the change in internal sound speed (m/μsec) comprises processing speed of at least about 0.3 frames per second.

In a related embodiment, the calculation of the change in internal sound speed (m/μsec) comprises processing speed of at least about 1.0 frames per second.

In another related embodiment, the calculation of the change in internal sound speed (m/μsec) comprises processing speed of at least about 2.0 frames per second.

In further related embodiment, the calculation of the change in internal sound speed comprises DSI algorithm processing speed between about 0.3 frames per second and about 3.0 frames per second, corresponding to increased speed of between about 2-fold and about 21-fold relative to a processing speed (frame per second) of a corresponding calculation of a change in strain (%) using Thermal Strain Imaging (TSI).

In some embodiments, the calculation of the change in internal sound speed is devoid of image processing/filtering; thereby allowing increasing processing speed of the calculation time of the change in internal sound speed.

According to some embodiments, the DSI algorithm comprises a first step of echo/pixel shift calculation that is estimated with dense optic flow, and a second step of determining the change in internal sound speed that is calculated as the solution to the inverse problem.

According to a specific embodiment, the inverse problem comprises regularized inverse problem.

In a specific embodiment, the acquisition of the at least two successive ultrasound (US) images comprise at least 20 frames per second.

The present disclosure provides methods and a system, including a computer-implemented method, for real-time non-invasive monitoring of a change in internal speed-of-sound in biological tissue, which can be used, for example, for temperature monitoring.

The monitoring is based on accurate sound deviation measurements executed during a treatment that is being performed on the tissue and inflicts a change on the underlying sound speed of the tissue and may also affect the internal temperature of the tissue.

The system and methods comprise collection/receival of at least two successively acquired ultrasound (US) images or raw radio frequency (RF) data thereof comprising an echo shift, of a target area of the biological tissue, and further comprise a speed sound/temperature estimating algorithm that accurately calculates the change in speed-of-sound between the acquired US images based on the echo shift, and then calculates/calibrates the change in the temperature of the tissue resulting from the measured speed-of-sound change.

Advantageously, feedback is provided regarding the treatment, such as HIFU (e.g. thermal ablation therapy). In some embodiments, the feedback comprises a recommendation to continue, to stop, or to adjust parameters of the treatment.

According to some aspects, there is provided a method of non-invasive monitoring of a change in internal sound speed of a biological tissue, comprising the steps of:
acquiring at least two successive ultrasound (US) images of a target area of a biological tissue during a treatment performed on the tissue, thereby obtaining raw radio frequency (RF) data of the acquired US images; and applying on the obtained data an algorithm that is configured to:
determining echo shift between the at least two successive ultrasound images; and calculating a change in the internal sound speed of the biological tissue based on the echo shift; and optionally estimating internal temperature change based on the calculated change in the internal sound speed; wherein the algorithm comprises a Dense Speed-of-Sound Shift Imaging (DSI) algorithm.

In some embodiments, the method further comprises an iteration of the steps of the method that allows real-time non-invasive monitoring of internal sound speed of a biological tissue and feedback on the treatment.

In some embodiments, the acquiring of at least two successive ultrasound (US) images comprises acquiring plane waves B-mode data.

In some embodiments, the acquiring of at least two successive ultrasound (US) images comprises acquiring at least nine plane waves or beams per image. Each possibility is a separate embodiment.

In some embodiments, the at least nine plane waves per image comprises at least three plane angles of about 5° intervals, and wherein each plane angle further comprises at least three angles selected at about 1.5° intervals.

In some embodiments, the acquiring of at least two successive ultrasound (US) images during the treatment is performed before, in parallel and/or after the treatment.

In some embodiments, the echo shift comprises a change/shift in speed-of-sound of the biological tissue.

In some embodiments, the treatment can inflict a physiological change on the inherent speed-of-sound of the biological tissue. In some embodiments, the treatment can inflict a physiological change in the internal temperature of the tissue.

In some embodiments, the treatment comprises mechanical procedure, non-mechanical procedure and/or non-invasive procedure.

In some embodiments, the treatment comprises high-intensity focused ultrasound (HIFU) thermal ablation therapy, cryo-ablation, adhesive bonding assessment, mechanical ablation, histotripsy, introducing a drug or other biological or chemical compound into the tissue, contrast agents, or polymer congealing.

In some embodiments, the treatment comprises high-intensity focused ultrasound (HIFU) thermal ablation therapy.

In some embodiments, the acquiring of the at least two successive ultrasound (US) images is performed from a separate transducer than the HIFU transducer.

In some embodiments, the thermal ablation therapy comprises tumor ablation. In some embodiments, the thermal ablation therapy comprises contrast agents.

In some embodiments, the contrast agent is selected from one of nanobubbles, nanodroplets, microbubbles, gas vesicles or combinations thereof.

In some embodiments, the biological tissue is in-vivo or ex-vivo. In some embodiments, the biological tissue is a tumor.

In some embodiments, the biological tissue comprises a soft tissue, solid tissue, fat, bone, cartilage, or chemical deposit, lymphatic vessel or a blood vessel.

According to some aspects, there is provided a system for non-invasive monitoring of a change in internal sound speed of a biological tissue, comprising:
at least one transducer capable of acquiring at least two successive ultrasound (US) images of a target area during a treatment performed on the tissue; a transmitter capable of beamforming obtained raw radio frequency (RF) data of the acquired US images; a processing unit capable of receiving the RF data and executing thereon an algorithm that calculates echo shift between two successive ultrasound images and calculate a change in internal sound speed based on the echo shift; and optionally, determining a change in internal temperature based on the change in the internal sound speed; and a monitor capable of presenting the change in the sound speed and/or temperature; and wherein the algorithm comprises or essentially consists of a Dense Speed-of-Sound Shift Imaging (DSI) algorithm. Each possibility is a separate embodiment.

In some embodiments, the transducer comprises a power output unit with a duty cycle of about 50%, frequency of about 2 MHz, and PRF of about 20 μsec.

In some embodiments, the transducer is capable of a plane steering acquisition control allowing acquisition of at least nine plane waves or beams per image. Each possibility is a separate embodiment.

In some embodiments, the at least nine plane waves per image comprises at least three plane angles of about 5° intervals, and wherein each plane angle further comprises at least three angles selected at about 1.5° intervals.

In some embodiments, the treatment comprises high-intensity focused ultrasound (HIFU) thermal ablation therapy.

In some embodiments, the acquisition of the at least two successive ultrasound (US) images is performed from a separate transducer than the HIFU transducer.

According to some aspects, there is provided a computer-implemented method for calculating a change in internal sound speed of a biological tissue, comprising: receiving raw radio frequency (RF) data of at least two successive acquired ultrasound (US) images of a target area; applying onto the received data an algorithm that is configured to:
measuring echo shift between the at least two successive ultrasound images;
calculating a change in sound speed based on the echo shift; and optionally calibrating the change in internal sound speed to determine a change in internal temperature; and providing an output comprising the change in the internal sound speed and/or temperature; and wherein the algorithm comprises or essentially consists of a Dense Speed-of-Sound Shift Imaging (DSI) algorithm. Each possibility is a separate embodiment.

In some embodiments, the raw radio frequency (RF) data of at least two successive acquired ultrasound (US) images comprises plane waves B-mode data.

In some embodiments, the raw radio frequency (RF) data of at least two successive acquired ultrasound (US) images comprises data of imaging of at least nine plane waves or beams per image. Each possibility is a separate embodiment.

In some embodiments, the at least nine plane waves per image comprises at least three plane angles of about 5° intervals, and wherein each plane angle further comprises at least three angles selected at about 1.5° intervals.

In some embodiments, the Dense Speed-of-Sound Shift Imaging (DSI) algorithm comprises a first step of translation calculation that is estimated with dense optic flow, and a second step of calculating the slowness deviation that is calculated as the solution to the regularized inverse problem.

In some embodiments, the Dense Speed-of-Sound Shift Imaging (DSI) algorithm comprises optical flow algorithm, spatial regularization constraints, and an inverse problem optimization.

Certain embodiments of the present disclosure may include some, all, or none of the above advantages. One or more technical advantages may be readily apparent to those skilled in the art from the figures, descriptions and claims included herein.

In addition to the exemplary aspects and embodiments described above, further aspects and embodiments will become apparent by reference to the figures and by study of the following detailed descriptions.

### BRIEF DESCRIPTION OF THE FIGURES

The patent or application file contains at least one drawing executed in color. Copies of this patent or patent application publication with color drawing(s) will be provided by the Office upon request and payment of the necessary fee

The invention will now be described in relation to certain examples and embodiments with reference to the following illustrative figures.

FIGS. 1A-1B schematically illustrates the steps of the method for non-invasive monitoring of a change in speed-of-sound and temperature. (i) acquiring ultrasound (US) B-Mode images at various plane wave steering angles; (ii) the pixel shift at consecutive time intervals is computed. This produces the measured Δd vector which is inverted to receive a change in sound slowness/speed Δv presented here as “solving the inverse problem” represented in equation 3. Optionally, the units of Δv may be converted to temperature by a pre-calibrated constant. The method facilitates real-time monitoring of a change in internal sound speed or internal temperature, thereby allowing feeding back on the treatment by providing the user adequate information/recommendation on how to proceed, for example with parameters related to the intensity or duration of the treatment being performed The method is performed during a treatment that affects the tissue's speed of sound or temperature, and involves applying a Dense Speed-of-Sound Shift Imaging algorithm (DSI algorithm) on the acquired US data, the DSI calculates the change in internal sound speed as the solution to the inverse problem which is represented in equation 3 thereby providing an advantageous, alternative solution to the round trip problem represented by commonly known equation 1.

FIG. 1C illustrates the round-trip problem. An area of the tissue with different speed-of-sound than the background is perceived as being translated along the axial direction of the plane wave. The left image illustrates homogenous tissue with an area of sound speed varying from the background (the area is illustrated as a round “object”) (could be a result of a treatment performed the tissue, such as thermal ablation or a pocket of microbubbles MBs); the following three images illustrate: three plane wave acquisitions steered at various angles (−5, 0, 5, for example) and the expected position of the varying area per steering angle, wherein each plane wave perceives the object as being slightly displaced due to the effect of equation 1. Reference is made to FIG. 5A illustrating the same during heating.

FIGS. 2A-2C simulate non-invasive monitoring of a change in speed-of-sound. The result of thermal strain imaging (TSI) is compared with the disclosed Dense Speed-of-Sound Shift Imaging algorithm (DSI) in agarose tissue-mimicking phantom.

FIG. 2A shows heatmaps of simulation results presenting the measured pixel shift (Left panel) for the ground truth method (i) the TSI algorithm (iii), and the DSI algorithm (v), and the reconstruction of the heated area/resulting slowness deviation (Right panel) for the for the ground truth method (ii) the TSI algorithm (iv), and the DSI algorithm (vi). Axes are common to subfigures (i)-(vi), color bar is common to subfigures (i), (iii), and (v).

FIGS. 2B-C show plots of simulation results presenting reconstruction cross-section analysis for the lateral (FIG. 2B) and axial (FIG. 2C) directions of the plane wave capturing lesion dimensions.

FIGS. 3A-3D show experimental results of non-invasive monitoring of a change in a speed-of-sound during using the DSI algorithm. The change in speed-of-sound was assessed ex-vivo on chicken breast biological tissue during thermal ablation (HIFU) (heating), and compared with the TSI method.

FIG. 3A illustrates the experimental setup.

FIG. 3B shows an image of a chicken breast tissue sample ex-vivo following treatment with thermal ablation for 270 s. White area is a necrotic tissue.

FIG. 3C shows heatmaps of experimental results presenting the measured pixel shift (Left panel) for the (i) TSI and (iii) DSI methods after the first treatment, and the resulting reconstruction of the pixel shift map of the treated region (Right panel) using (ii) TSI and (iv) DSI methods. Axes are common to subfigures (i)-(iv). Color bar is common to subfigures (i) and (iii).

FIG. 3D shows heatmaps presenting the effect of treatment duration using the DSI algorithm. HIFU treatment was conducted for 45 s, 90 s, or 135 s, yielding the pixel shifts (Left panel; (i), (iii), and (v), respectively) and the sound slowness (Right panel; (ii), (iv), (vi), respectively). Axes are common to all subfigures. Color bar is common to subfigures (i), (iii), and (v), and to (ii), (iv), and (vi).

FIGS. 4A-4B presents estimation of temperature change based on the change in speed-of-sound.

FIG. 4A shows box & whisker chart of the slowness deviation analyzed across several experiments (N=6 samples) using DSI to measure the effect of treatment time on speed-of-sound shift.

FIG. 4B shows a line graph presenting comparison of temperature estimation as a function of HIFU duration (N=6 samples) using Ground truth thermocouple measurements (blue line), TSI (orange line), and DSI (green line). Estimation of temperature change using the DSI algorithm is calculated based on the speed-of-sound shift calculated in FIG. 4A, and using the pre-calibration alpha factors extracted by comparing the results to the thermocouple (ground-truth). Results are presented as mean±std.

FIGS. 5A-5C present experimental results of a trend in the result of the disclosed algorithm (DSI) during HIFU including 7 treatments of thermal ablation.

FIG. 5A illustrates the round-trip problem. When the internal speed-of-sound of an area of a tissue changes due to heating that is applied on the tissue, that area of the tissue with different speed-of-sound than the background is perceived as being translated along the axial direction of the plane wave. The sound wave round trip time is illustrated assuming a homogeneous sound speed throughout the field of view (left), so when heating is applied (middle) the sound speed changes locally, and the same object yields a different round trip time (right). The disclosed DSI provides the solution to the inverse problem, represented by equation 3, as an alternative advantageous approach for solving the round-trip problem/echo shift problem.

FIG. 5B heatmap of the optical flow measurements during HIFU after 1, 3, 5, and 7 thermal ablation treatments.

FIG. 5C heatmaps of sound slowness deviation measurements during HIFU after 1, 3, 5, and 7 thermal ablation treatments.

FIG. 6 shows experimental results of real time non-invasive monitoring of a change in a temperature using the disclosed algorithm (DSI). The change in temperature was assessed during cryo-ablation (cooling) performed ex-vivo on chicken breast biological tissue.

FIGS. 7A-7B shows experimental results of real time non-invasive monitoring of a change in a temperature using the disclosed algorithm (DSI). The change in temperature was induced by microbubbles (MB) contrast agents and assessed in tissue-mimicking phantoms.

FIG. 7A presents the results of the pixel shift (i-iv) and the reconstructed change in speed-of-sound (v-viii), compared to standard contrast enhanced imaging (ix). Deviations in speed-of-sound were induced by four MB concentrations of 0.87 MB/ml, 1.25 MB/ml, 1.75 MB/ml, and 2.92×106 MB/ml.

FIG. 7B shows line graphs presenting quantification of the results of FIG. 7A including the four MB concentrations. Shown are contrast ratio (blue circles), maximal pixel shifts (red squares), and maximal speed of sound shift (purple triangles). A clear relationship is shown between the MB concentration and contrast attained by the DSI algorithm.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Unless otherwise defined, all technical and scientific terms used herein have the same meaning as commonly understood by one of ordinary skill in the art to which this disclosure pertains.

As used herein, the singular forms “a”, “an” and “the” are intended to include the plural forms as well, unless the context clearly indicates otherwise. “a” and “an” are used herein to refer to one or more than one (i.e., to at least one) of the stated object, unless the context clearly dictates otherwise. By way of example, “a treatment” means one or more treatments.

As used herein, the term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “may” refer to an optional or possible, approach or possibility, but not a requirement. The term “can” refer to a permissible or plausible, approach or possibility, but not a requirement.

As used herein, “optional” or “optionally” means that the subsequently described event or circumstance does or does not occur, and that the description includes instances where said event or circumstance occurs and instances where it does not.

As used herein, the term “comprising” is synonymous with the terms “including,” “containing,” or “characterized by,” and is inclusive or open-ended i.e. does not exclude additional, unrecited elements. According to some embodiments, the term comprising may be replaced with the term with the term “consisting of” which excludes any element, step, or ingredient not specified in the claim. According to some embodiments, the term comprising may be replaced with the term “consisting essentially of” which limits the scope of a claim to the specified materials or steps “and those that do not materially affect the basic and novel characteristics” of the claimed invention.

The term “treatment” as used herein refers to both therapeutic treatment and prophylactic or preventative measures, including for example medical intervention in the form of pharmaceuticals or surgery or ablation therapy. In some embodiments, those in need of treatment include those already having a condition as well as those in which the condition is to be prevented.

The present disclosure provides systems and methods, including a computer-implemented method for real-time non-invasive monitoring of a change in internal speed-of-sound of a biological tissue which can be calibrated to evaluate a change in internal temperature of the tissue.

In some embodiments, these methods and systems are useful for monitoring biological applications and prevent damage to the tissue, for example, by inducing necrosis, cavitation and/or heat shock to the cells, or any combination thereof.

According to one aspect, there is provided a method for non-invasive monitoring of a change in internal sound speed of a biological tissue indicative of a treatment, comprising the steps of: (a) acquiring at least two successive ultrasound (US) images of a target area of a biological tissue, thereby obtaining raw radio frequency (RF) data of the acquired US images; and (b) applying onto the obtained data a Dense Speed-of-Sound Shift Imaging (DSI) algorithm configured to: (i) determine echo/pixel shift (μm) between the at least two successive ultrasound images; and (ii) calculate a change in the internal sound speed (m/μsec) of the biological tissue based on the echo/pixel shift;
and wherein the DSI algorithm is characterized by solving an inverse problem; and wherein the calculated change in the internal sound speed of the biological tissue comprises the solution to the inverse problem comprises. Each possibility is a separate embodiment.

According to some embodiments, the method includes iterations of the steps of the method (at least steps (a)-(b)) thereby allowing real-time non-invasive monitoring of a change in the internal sound speed of the biological tissue.

According to some embodiments, the method includes a step (c) of feeding back on the treatment; the feedback comprises providing a user with the calculated change in the inherent speed-of-sound of the biological tissue.

According to some embodiments, the method includes a step (c) of feeding back on the treatment; the feedback comprises providing a user with a recommendation(s) at least about terminating or continuing the treatment and/or about adjusting treatment parameters. Each possibility is a separate embodiment.

According to some embodiments, the method includes iterations of the steps of the method (at least steps (a)-(b) and step (c) feeding back) thereby allowing real-time non-invasive monitoring of a change in the internal sound speed of the biological tissue.

In some embodiments, treatment parameters include parameters such as, but not necessarily limited to, treatment intensity and/or treatment duration.

As used herein, the terms “sound speed” and “speed-of-sound” (e.g., in microseconds per meter) may be interchangeably used and refer to the time it takes for acoustic/sound waves to travel a distance, internally, in a biological tissue.

The “sound speed” is related to the term “sound slowness” by an inverse ratio (sound speed=1/sound slowness). The term “sound slowness” is represented in equation 4, where it is referred to as the solution to the advantageous inverse problem which is presented in equation 3.

As used herein, the terms “sound speed” and “sound slowness” may be used interchangeably, particularly when referring to the change/deviation in sound speed or change/deviation in sound slowness which ought to be the same.

As used herein, the term “Echo Shift” (e.g., in millimeter) may refer to, or include, the perceived/measured deviation between two representative acoustic wave returning from a tissue (i.e., change in round-trip), wherein one of the acoustic wave is indicative of a treatment applied on the tissue, while the second wave returning, for example, from an untreated area (i.e., background) or from the same area after the treatment has terminated or adjusted.

The term “Pixel shift” is related to term “echo shift” and refers to perceived/measured deviation between corresponding pixels in two successive ultrasound images.

In the art the term “echo shift” is related to the term “round-trip problem” and has a representation in equation 1 which represents the round-trip problem in its classical form.

The herein disclosed DSI algorithm offers calculating the pixel shift as part of an alternative solution to the round-trip problem, a solution that determines the “sound slowness” (equation 4) and is received when solving the advantageous inverse problem represented by the herein disclosed equation 3.

In accordance, in some of the embodiments of the present disclosure the terms “Echo Shift” and “Pixel shift” may be used interchangeably.

The terms “change”, “shift” and “deviation” are also interchangeable.

It is known in the art that some medical procedures and treatments performed on biological tissue, such as thermal ablation, cryo-ablation, use of contrast agents, and others, affect the speed of sound waves as they travel within the treated area of the biological tissue. The extent of the effect of the treatment (i.e., the change in speed-of-sound due to the treatment) can be calculated based on the Echo Shift.

As used herein, the term “acquiring at least two successive ultrasound (US) images” refers to obtaining/collecting/receiving raw radio frequency (RF) data in a sequential manner over time during a treatment. Successive raw radio frequency (RF) data may be collected at a rate of about 20 frames per second and up to 1000 frames per second, and may be further collected at different time points during the medical treatment procedure, or sessions thereof.

The terms “successive” and “sequential” are used interchangeably to refer to the temporal aspect of the data.

It is to be understood that the “acquiring” of successive images may include collection of many hundreds or many thousands of such frames, or at least two of them.

In some embodiments, successive ultrasound (US) images comprise at least 2 US frames/images, at least 20 US frames/images, at least 200 US frames/images, at least 2000 US frames/images, at least 10,000 US frames/images, or more. Each possibility is a separate embodiment. The terms “images” and “frames” are interchangeable.

The analysis of that data includes determining the change/shift in echo/pixel between the at least two successive ultrasound images, by comparing between two frames/images each time a comparison made. A comparison may include a combination of any two frames/images of the at least two successive ultrasound images, optimally, the two frames/images are temporally close to each other for obvious temporal resolution reasons.

In some embodiments, the method for non-invasive monitoring of a change in internal sound speed of a biological tissue indicative of a treatment, comprises the steps of (a) acquiring at least two successive ultrasound (US) images of a target area of a biological tissue, thereby obtaining raw radio frequency (RF) data of the acquired US images.

In some embodiments, the method for non-invasive monitoring of a change in internal sound speed of a biological tissue indicative of a treatment, comprises the step of (b) applying onto the obtained data a Dense Speed-of-Sound Shift Imaging (DSI) algorithm.

In some embodiments, the DSI algorithm is configured to (i) determine echo/pixel shift (μm) between the at least two successive ultrasound images.

In some embodiments, the DSI algorithm is configured to (ii) calculate a change in the internal sound speed (m/μsec) of the biological tissue based on the echo/pixel shift.

In some embodiments, the DSI algorithm is characterized by solving an inverse problem; in some embodiments, a solution to the inverse problem includes the calculated change in the internal sound speed of the biological tissue; in some embodiments, the change in internal sound speed that is calculated as the solution to the inverse problem.

In some embodiments, the inverse problem includes a regularized inverse problem.

In some embodiments, the DSI algorithm comprises a first step of echo/pixel shift calculation that is estimated with dense optic flow, and a second step of determining the change in internal sound speed that is calculated as the solution to the inverse problem.

Reference is now made to the theoretical part elsewhere hereinbelow presenting equations 1-7, particularly to equation 3 representing the inverse problem and equation 4 presenting the solution referred to as the sound slowness, and to equation 6 representing concept for solving the inverse problem and equation 7 presenting the regularized solution referred to as the sound slowness.

In some embodiments, the DSI algorithm includes equation 3 representing the inverse problem, and equation 4 presenting the solution referred to as the sound slowness.

In some other embodiments, the DSI algorithm includes equation 3 representing the inverse problem, and equation 4 presenting the solution referred to as the sound slowness; and further includes equation 6 representing concept for solving the inverse problem and equation 7 presenting the regularized solution for the inverse problem referred to as the sound slowness.

In some embodiments, the DSI algorithm is characterized by a solution for the inverse problem. In some embodiments, the inverse problem comprises a regularized inverse problem.

In some embodiments, a solution to the inverse problem of equation 3 comprises the solution described by equation 7.

In some embodiments, the method includes a step of estimating internal temperature change of the biological tissue based on the calculated change in the internal sound speed of the biological tissue.

The estimation of internal temperature change of the biological tissue based on the calculated change in the internal sound speed is a step in the method, and is not necessarily part of the DSI algorithm.

In some embodiments, the step of estimating internal temperature change is performed by a different algorithm than the DSI algorithm. In some embodiments, the algorithm encompasses the DSI algorithm. In some embodiments, the different algorithm encompasses the DSI algorithm and is configured to provide the temperature estimation and the feedback with recommendation(s) to a user.

In some other embodiments, the DSI can calculate the sound slowness (Equation 4) and then convert to temperature (Equation 5).

In some related embodiments, the estimation of internal temperature change includes converting sound slowness units to temperature by a pre-calibrated constant.

In some embodiments, sound slowness (μsec/m) is the inverse of speed sound (m/μsec) (i.e., 1/speed sound).

As used herein, the term “pre-calibration constant” relates to the implementation of the temperature conversion. The result of Equation 4 (Av) is linked by Equation 5 to temperature by a constant. The pre-calibration constant is an empirical value connecting between changes in sound slowness and temperature changes. To acquire the pre-calibration constant, the sound speed change is estimated by the DSI algorithm while a thermocouple measures the true temperature. Then, according to some embodiments, the constant is calculated by fitting these two results (sound slowness and temperature) to Equation 5 in the case of DSI (or 2, in the case of TSI). In some embodiments, the result is the variable referred to as alpha.

In some embodiments, in water-based soft tissue, the constant α1 comprises 0.1%° C.⁻¹ change in thermal strain for a 1° C. increase in temperature.

In some embodiments, in temperatures up to 40° C., the relationship between sound speed and temperature change is mostly linear and α2 should correspond to a change of up to 1 ms⁻¹ for a 1° C. rise in temperature, depending on the target tissue.

In some embodiments, the DSI algorithm determines a temperature change between 3° C. and 15° C. with high thermometry precision of less than 2° C. error for temperature changes as low as 8° C.

According to one aspect, there is provided a method for non-invasive monitoring of a change in internal sound speed of a biological tissue indicative of a treatment, comprising the steps of: (a) acquiring at least two successive ultrasound (US) images of a target area of a biological tissue, thereby obtaining raw radio frequency (RF) data of the acquired US images; and (b) applying onto the obtained data a Dense Speed-of-Sound Shift Imaging (DSI) algorithm configured to: (i) determine echo/pixel shift (μm) between the at least two successive ultrasound images; and (ii) calculate a change in the internal sound speed (m/μsec) of the biological tissue based on the echo/pixel shift; and (c) estimating internal temperature change of the biological tissue based on the calculated change in the internal sound speed of the biological tissue; and
wherein the DSI algorithm is characterized by solving an inverse problem; and wherein the calculated change in the internal sound speed of the biological tissue comprises the solution to the inverse problem comprises.

According to some embodiments, the method includes iterations of the steps of the method (at least steps (a)-(b) and step (c) of temperature estimation) thereby allowing real-time non-invasive monitoring of a change in the internal sound speed of the biological tissue and/or a change in the internal temperature of the biological tissue. Each possibility is a separate embodiment.

According to some embodiments, the method includes iterations of the steps of the method (at least steps (a)-(b)) and step (c) of temperature estimation and step (d) of feeding back) thereby allowing real-time non-invasive monitoring of a change in the internal sound speed of the biological tissue and/or a change in the internal temperature of the biological tissue. Each possibility is a separate embodiment.

According to some embodiments, the method includes a step of feeding back on the treatment; the feedback comprises providing a user with the calculated change in the inherent speed-of-sound of the biological tissue and/or with the calculated change in the internal temperature of the biological tissue. Each possibility is a separate embodiment.

According to some embodiments, the method includes a step of feeding back on the treatment; the feedback comprises providing a user with a recommendation(s) at least about terminating or continuing the treatment and/or about adjusting treatment parameters. Each possibility is a separate embodiment.

In some embodiments, treatment parameters include parameters such as, but not necessarily limited to, treatment intensity and/or treatment duration. Each possibility a separate embodiment.

According to some embodiments, the acquisition of at least two successive ultrasound (US) images of a target area of a biological tissue is being performed during a treatment on the tissue thereby providing indication about the treatment.

In some specific embodiments, during a treatment includes at least in parallel to the treatment, and optionally before and/or after applying the treatment on the tissue. Each possibility is a separate embodiment.

In some specific embodiments, during a treatment includes at least in parallel to the treatment.

In some specific embodiments, during a treatment includes immediately before applying the treatment, and in parallel to the treatment.

In some specific embodiments, during a treatment includes at least in parallel to the treatment and after applying the treatment on the tissue; wherein according to some embodiments, the after refers to, and include, any time after the treatment, or part thereof, or sessions thereof, that may be indicative of the treatment that was performed.

As used herein, the term “indicative of the treatment” may refer to a change in the inherent speed-of-sound and/or internal temperature of a tissue as a consequence of a treatment applied thereon.

In other words, receiving indication on the treatment refers to the possibility of monitoring the tissue's internal speed-of-sound and/or internal temperature based on the acquired US images of the tissue. Such indication about the treatment may be received as a consequence of applying the treatment, or as a consequence of terminating the treatment or adjusting its parameters.

According to a related embodiment, the treatment can inflict a physiological change on the inherent speed-of-sound of the biological tissue. According to a related embodiment, the treatment can inflict a physiological change in the internal temperature of the biological tissue.

According to some embodiments, the treatment can inflict a physiological change on the inherent speed-of-sound of the biological tissue and/or the treatment can inflict a physiological change in the internal temperature of the biological tissue. Each possibility a separate embodiment.

In some embodiments, the acquisition of at least two successive ultrasound (US) images comprises acquiring plane waves B-mode data.

In some embodiments, the acquisition of at least two successive ultrasound (US) images comprises acquiring at least nine plane waves or beams per imaging sequence. Each possibility is a separate embodiment.

In some embodiments, the at least nine plane waves per imaging sequence comprises at least three plane angles of about 5° intervals, and wherein each plane angle further comprises at least three angles selected at about 1.5° intervals.

As a non-limiting example, the at least three plane angles of about 5° intervals may include [−5°, 0°, 5°].

As a non-limiting example, a plane angle further including at least three angles selected at about 1.5° intervals may include about [6.5°, 5°, 3.5°], or about [−1.5°, 0°, 1.5°], or about [−6.5°, −5°, −3.5°].

The disclosed method provides a surprising advantage in the average runtime it takes for the DSI algorithm to calculate the change in the internal speed-of-sound.

In some embodiments, the calculation of the change in internal sound speed (m/μsec) comprises DSI algorithm processing speed of at least about 0.2 frames per second.

In some embodiments, the calculation of the change in internal sound speed (m/μsec) comprises DSI algorithm processing speed of at least about 0.3 frames per second.

In some embodiments, the calculation of the change in internal sound speed (m/μsec) comprises DSI algorithm processing speed of at least about 3.0 frames per second.

In some embodiments, the calculation of the change in internal sound speed comprises DSI algorithm processing speed of at least about 0.2 frames per second, at least about 0.3 frames per second, at least about 0.4 frames per second, at least about 0.5 frames per second, at least about 0.6 frames per second, at least about 0.7 frames per second, at least about 0.8 frames per second, at least about 0.9 frames per second, at least about 1.0 frames per second, at least about 1.2 frames per second, at least about 1.5 frames per second, at least about 2.0 frames per second, at least about 2.5 frames per second, at least about 3.0 frames per second, or more. Each possibility is a separate embodiment.

In some embodiments, the calculation of the change in internal sound speed comprises DSI algorithm processing speed of between about 0.2 frames per second and about 3.0 frames per second, or between about 0.3 frames per second and about 3.0 frames per second, or between about 0.5 frames per second and about 3.0 frames per second, or between about 1.0 frames per second and about 3.0 frames per second, or between about 2.0 frames per second and about 3.0 frames per second. Each possibility is a separate embodiment.

In some embodiments, the calculation of the change in internal sound speed comprises DSI algorithm processing speed of between about 0.2 frames per second and about 3.0 frames per second. In some embodiments, the calculation of the change in internal sound speed comprises DSI algorithm processing speed of between about 0.3 frames per second and about 3.0 frames per second.

In some embodiments, the calculation of the change in internal sound speed comprises DSI algorithm processing speed of between about 0.3 frames per second and about 3.0 frames per second, and corresponds to increase in the speed (frame per second) of the calculation of the change in internal sound speed of between at least about 2-fold (about 0.3 frames per second) and about 20/22-fold (about 3.0 frames per second).

The herein disclosed average runtime refers to the mean time it takes for each of DSI or TSI to process a frame thereby to calculate internal speed-of-sound or strain, respectively, i.e., time per frame or the inverse thereof frame per time.

In some embodiment, the average runtime of calculating strain with the Thermal Strain Imaging (TSI) algorithm was 7.21±0.5 seconds/frame (equivalent to about 0.14±0.1 frames per second, rounded to about 0.15 frames per second); while in some embodiments, the average runtime of DSI for calculating the change in internal speed-of-sound was 0.33±0.004 seconds/frame (equivalent to about 3.0 frames per second). Each possibility is a separate embodiment.

In some embodiment, a speedup of about ×20 or about ×21.5 was achieved with DSI relative to TSI.

In some embodiment, the DSI algorithm provides an increased speed (frame per second) in the calculation time of the change in internal sound speed of at least about 2-fold relative to a calculation time (frame per second) of a corresponding strain using Thermal Strain Imaging (TSI) (or 0.3 frames per second relative to 0.15 frames per second).

In some embodiment, the DSI algorithm provides an increased speed (frame per second) in the calculation time of the change in internal sound speed of at least about 2-fold, or at least about 4-fold, or at least about 8-fold, or at least about 10-fold, or at least bout 12-fold, or at least about 16-fold, or at least about 20-fold (or 3.0 frames per second relative to 0.15 frames per second), relative to a calculation time (frame per second) of a corresponding strain using Thermal Strain Imaging (TSI). Each possibility is a separate embodiment.

In some embodiment, the DSI algorithm provides an increased speed (frame per second) in the calculation time of the change in internal sound speed of between at least about 2-fold (about 0.3 frames per second) and about 21.5-fold (about 3.0 frames per second), relative to a calculation time (frame per second) of a corresponding strain using Thermal Strain Imaging (TSI) (about 0.15 frames per second).

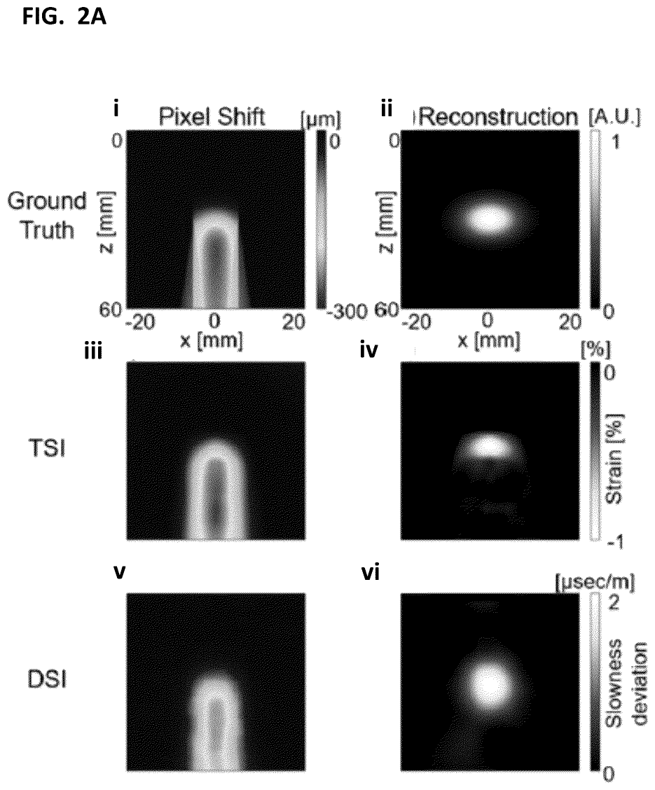

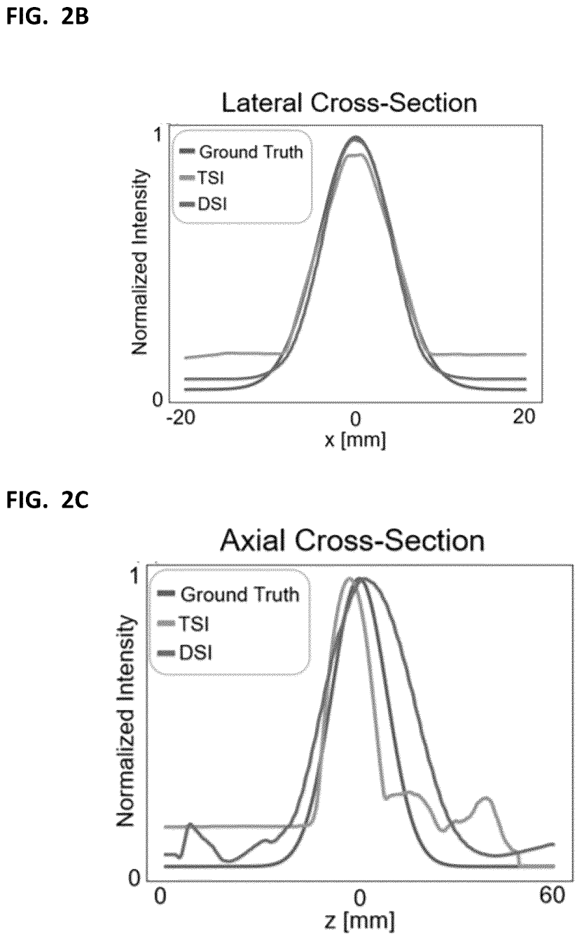

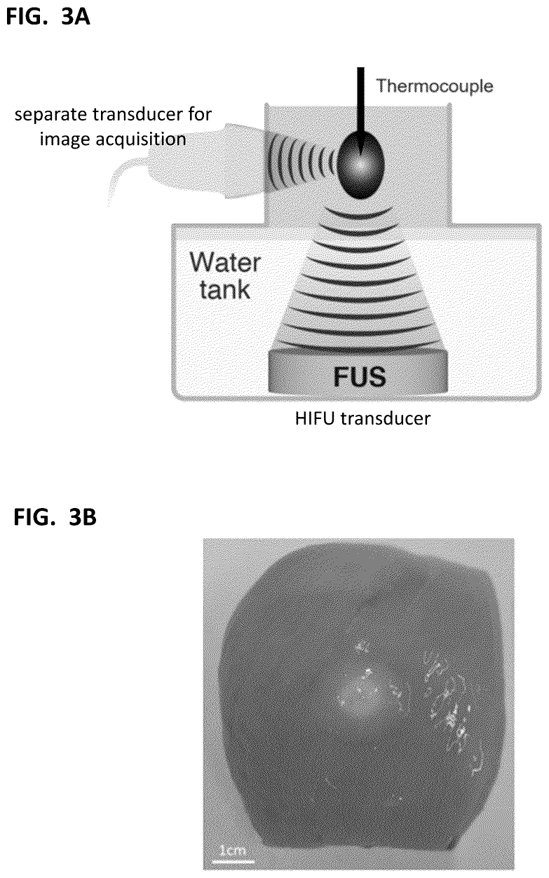

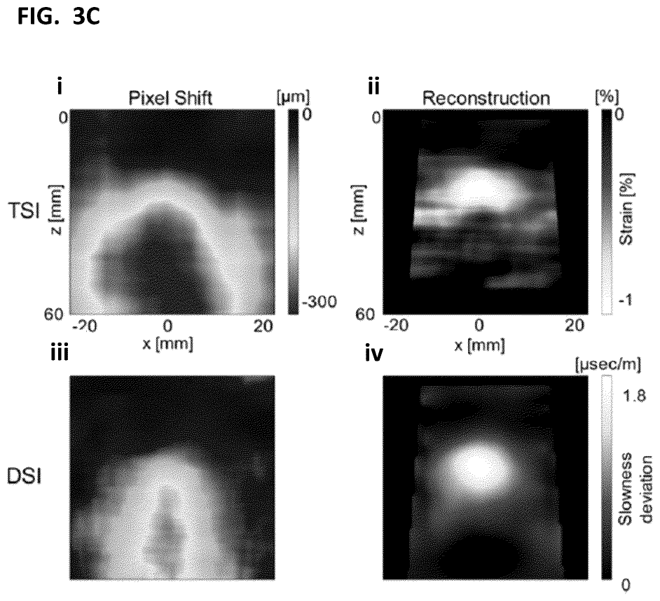

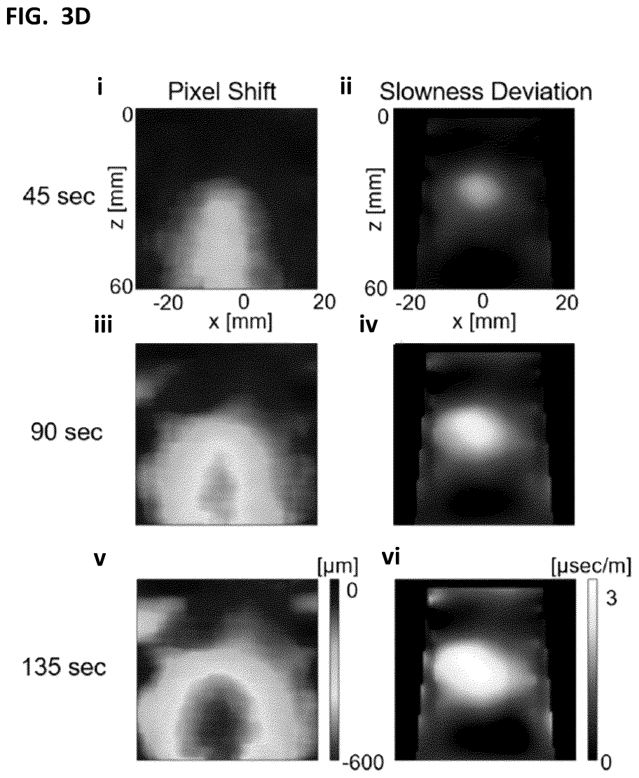

Reference is made to Example 6 exemplifying speed performance of DSI, also in comparison to TSI, and in accordance with the abovementioned embodiments. Reference is also made to Example 2, FIGS. 2A-2C and Example 3, FIGS. 3C-3D which demonstrate calculation of speed of sound change at a speed of 3.0 frames per second.

The disclosed method provides the advantage of the DSI algorithm being devoid of image processing/filtering. According to some embodiments, the DSI algorithm is devoid of image processing/filtering. This is attributed to the DSI algorithm using all of the available pixels simultaneously rather than calculating values for individual pixels and smoothing them together. Removal of the additional image filtering step relieves computations thus reducing the overall computation time.

In some embodiments, the DSI algorithm uses all of the available pixels simultaneously. In some embodiments, the DSI algorithm uses all of the available pixels simultaneously, compared to the TSI method that calculates values for individual pixels and then integrates them together.

Reference is made to FIG. 2 (iv) and FIG. 2(vi); FIG. 3C(ii) and FIG. 3C(iv) comparing reconstruction of a change in strain using TSI algorithm having average calculation speed of about 0.15 frames per second vs. reconstruction of a change in speed sound using DSI algorithm devoid of image processing/filtering having average calculation speed of about 3.0 frames per second.

According to some embodiments of the method the calculation of a change in the internal sound speed of the biological tissue comprises determining a size of a focal area of a lesion; and wherein the DSI algorithm determines the size of a focal area (mm) of a lesion as having a reduced size relative to a size of a focal area (mm) of a same lesion determined by Thermal Strain Imaging (TSI).

In some embodiments, determining the size of a focal area (mm) of a lesion as having a reduced size relative to a size of a focal area (mm) of a same lesion determined by Thermal Strain Imaging (TSI) includes reduction in FWHM in the lateral direction.

In some embodiments, determining the size of a focal area (mm) of a lesion as having a reduced size relative to a size of a focal area (mm) of a same lesion determined by Thermal Strain Imaging (TSI) includes reduction in FWHM in the lateral direction; and wherein reduction in FWHM in the lateral direction includes improved spatial coherence relative to TSI.

In some specific embodiments, the DSI algorithm determines the size of a focal area (mm) of a lesion as having at least about 10% reduction in size relative to a size of a focal area (mm) of a same lesion determined by TSI. In some embodiments, the reduction includes or consists of reduction in FWHM in the lateral direction. Each possibility is a separate embodiment. In some embodiments, reduction in FWHM in the lateral direction is indicative of improved spatial coherence.

In some specific embodiments, the DSI algorithm determines the size of a focal area (mm) of a lesion as having between about 10% and about 20% reduction in size relative to a size of a focal area (mm) of a same lesion determined by TSI. Each possibility is a separate embodiment. In some embodiments, the reduction includes reduction in FWHM in the lateral direction. In some embodiments, reduction in FWHM in the lateral direction is indicative of improved spatial coherence.

In some embodiments, the reconstructed change in the internal sound speed (m/μsec) of the biological tissue includes improved spatial coherence relative a reconstructed strain (%) using state-of-the-art TSI. In some embodiments, the DSI algorithm is characterized by improved spatial coherence relative a reconstructed strain (%) using state-of-the-art TSI. In some embodiments, improved spatial coherence is characterized by reduction in FWHM in the lateral direction. In some embodiments, improved spatial coherence includes reduction in FWHM in the lateral direction. In some embodiments, the DSI algorithm is characterized by determining the size of a focal area (mm) of a lesion as having a reduced size relative to a size of a focal area (mm) of a same lesion determined by Thermal Strain Imaging (TSI). In some embodiments, improved spatial coherence includes determining the size of a focal area (mm) of a lesion as having a reduced size relative to a size of a focal area (mm) of a same lesion determined by Thermal Strain Imaging (TSI).

In some embodiments, the DSI algorithm is characterized by reconstructed change in internal speed-of-sound having improved spatial coherence relative to Thermal Strain Imaging (TSI); said improved spatial coherence comprises a reduction in FWHM in the lateral direction, relative to TSI; the reduction in FWHM in the lateral direction includes, or consist of, reduction in the determined size of a focal area (mm) of a lesion relative to a size of a focal area (mm) of a same lesion determined by Thermal Strain Imaging (TSI); and wherein the reduction comprises at least about 10% reduction relative to TSI.

In some embodiments, the treatment comprises mechanical procedure, non-mechanical procedure and/or non-invasive procedure, or any combination thereof. Each possibility is a separate embodiment.

In some embodiments, the treatment comprises one or more treatments selected from: high-intensity focused ultrasound (HIFU) thermal ablation therapy, cryo-ablation, adhesive bonding assessment, mechanical ablation, histotripsy, introducing a drug or other biological or chemical compound into the tissue, contrast agents, and polymer congealing, or any combination thereof; and wherein said treatment can inflict a physiological change on the inherent speed-of-sound and/or the internal temperature of the biological tissue. Each possibility is a separate embodiment.

In some embodiments, the treatment comprises one or more treatments selected from: high-intensity focused ultrasound (HIFU) thermal ablation therapy, cryo-ablation, and contrast agents, or any combination thereof. Each possibility is a separate embodiment.

In some embodiments, the treatment comprises high-intensity focused ultrasound (HIFU) thermal ablation therapy. In some embodiments, the treatment comprises cryo-ablation. In some embodiments, the treatment comprises use of contrast agents.

In some embodiments, the acquiring of the at least two successive ultrasound (US) images is performed from a separate transducer than the HIFU transducer. Reference is made to FIG. 3A.

In some embodiments, the thermal ablation therapy comprises tumor ablation. In some embodiments, the thermal ablation therapy comprises contrast agents.

In some embodiments, the contrast agent is selected from one or more of nanobubbles, nanodroplets, microbubbles, and gas vesicles, or any combination thereof. Each possibility is a separate embodiment.

In some embodiments, the method for non-invasive monitoring of a change in internal sound speed of is performed on a biological tissue in-vivo or ex-vivo providing indication of a treatment performed biological tissue.

In some embodiments, the biological tissue comprises in-vivo biological tissue and/or ex-vivo biological tissue. In some embodiments, the treatment is performed on a biological tissue in-vivo or ex-vivo. Each possibility is a separate embodiment. In some embodiments, the biological tissue comprises a tumor.

In some embodiments, the biological tissue comprises one or more tissue selected from a soft tissue, solid tissue, fat, bone, cartilage, or chemical deposit, lymphatic vessel or a blood vessel, or any combination thereof. Each possibility is a separate embodiment.

According to another aspect, the present disclosure provides a system for real-time non-invasive monitoring of a change in internal sound speed of a biological tissue, comprising: (a) at least one transducer capable of acquiring at least two successive ultrasound (US) images of a target area of a biological tissue; (b) a transmitter capable of beamforming obtained raw radio frequency (RF) data of the acquired US images; (c) a processing unit capable of receiving the RF data and executing thereon a Dense Speed-of-Sound Shift Imaging (DSI) algorithm configured to: (i) determine echo/pixel shift (μm) between the at least two successive ultrasound images; and (ii) calculate a change in the internal sound speed (m/μsec) of the biological tissue based on the echo/pixel shift; and (d) an output device capable of presenting the calculated change in the sound speed of the biological tissue; and
wherein the DSI algorithm is characterized by solving an inverse problem; and wherein the calculated change in the internal sound speed of the biological tissue comprises the solution to the inverse problem. Each possibility is a separate embodiment.

According to some embodiments, the processing unit is configured to feedback on the treatment; the feedback comprises providing a user with a recommendation(s) at least about terminating or continuing the treatment and/or about adjusting treatment parameters.

In some embodiments, the recommendation(s) are presented to the user by the same or different output device that is capable of presenting the calculated change in the sound speed of the biological tissue.

In some embodiments, the output device presenting the calculated change in the sound speed of the biological tissue and the output device providing a user with a recommendation(s) is same or different output device.

In some embodiments, treatment parameters include parameters such as, but not necessarily limited to, treatment intensity, and/or treatment duration. Each possibility is a separate embodiment.

As used herein, the term “output device” refers to a device capable of presenting information, such as but not limited to a computer monitor that can display information in pictorial or textual form.

In some embodiments, the output device can receive information from the processing unit. In some embodiments, the output device comprises a monitor.

According to some embodiments, the system comprises (a) at least one transducer capable of acquiring at least two successive ultrasound (US) images of a target area of a biological tissue.

According to some embodiments, the system comprises (b) a transmitter capable of beamforming obtained raw radio frequency (RF) data of the acquired US images.

According to some embodiments, the system comprises (c) a processing unit capable of receiving the RF data and executing thereon a Dense Speed-of-Sound Shift Imaging (DSI) algorithm configured to: (i) determine echo/pixel shift between the at least two successive ultrasound images; and (ii) calculate a change in the internal sound speed of the biological tissue based on the echo/pixel shift.

According to some embodiments, the system comprises (d) an output device capable of presenting the calculated change in the sound speed of the biological tissue.

According to some embodiments of the system, the DSI algorithm is characterized by solving an inverse problem; and wherein a solution to the inverse problem comprises the calculated change in the internal sound speed comprises. Each possibility is a separate embodiment.

In some embodiments, the processing unit is configured to estimate internal temperature change of the biological tissue based on the calculated change in the internal sound speed of the biological tissue.

In some related embodiments, the processing unit is configured to provide a presentation of the calculated change in temperature on the output device.

In some related embodiments, the estimation of internal temperature change comprises converting sound slowness units to temperature by a pre-calibrated constant.

In some embodiments, sound slowness (μsec/m) is the inverse of speed sound (m/μsec) (i.e., 1/speed sound).

According to another aspect, the present disclosure provides a system for real-time non-invasive monitoring of a change in internal sound speed of a biological tissue, comprising: (a) at least one transducer capable of acquiring at least two successive ultrasound (US) images of a target area of a biological tissue; (b) a transmitter capable of beamforming obtained raw radio frequency (RF) data of the acquired US images; (c) a processing unit capable of receiving the RF data and executing thereon a Dense Speed-of-Sound Shift Imaging (DSI) algorithm configured to: (i) determine echo/pixel shift between the at least two successive ultrasound images; and (ii) calculate a change in the internal sound speed of the biological tissue based on the echo/pixel shift; wherein the processing unit is further configured to estimate internal temperature change of the biological tissue based on the calculated change in the internal sound speed of the biological tissue; and (d) an output device capable of presenting the calculated change in the sound speed of the biological tissue and/or a change in the internal temperature of the biological tissue; and
wherein the DSI algorithm is characterized by solving an inverse problem; and wherein a solution to the inverse problem comprises the calculated change in the internal sound speed comprises. Each possibility is a separate embodiment.

In some embodiments, the processing unit is configured to calculate a change in internal temperature of the biological tissue by executing on the calculated internal sound speed a different algorithm than the DSI algorithm; the algorithm may encompass the DSI.

In some embodiments, the step of estimating internal temperature change is performed by a different algorithm than the DSI algorithm. In some embodiments, the algorithm encompasses the DSI algorithm. In some embodiments, the algorithm encompasses the DSI algorithm is configured to provide temperature estimation and feedback with recommendation(s) to a user.

In some embodiments, the acquisition of at least two successive ultrasound (US) images of a target area of a biological tissue is performed during a treatment performed on the tissue thereby providing indication about the treatment.

In some embodiments, the treatment can inflict a physiological change on the inherent speed-of-sound of the biological tissue and/or wherein the treatment can inflict a physiological change in the internal temperature of the biological tissue. Each possibility is a separate embodiment.

In some embodiments, the during a treatment comprises at least in parallel to the treatment. In some embodiments, the during a treatment comprises at least in parallel to the treatment, and before applying the treatment on the tissue and/or after applying the treatment on the tissue. Each possibility is a separate embodiment.

In some embodiments, after applying the treatment on the tissue includes any time after the treatment, or part thereof, or sessions thereof, that may be indicative of a change in the speed-of-sound and/or a change in internal temperature.

In some embodiments, the time after the treatment, or part thereof, or sessions thereof, that may be indicative of a change in the speed-of-sound and/or a change in internal temperature may vary based on the type of treatment being applied.

In some embodiments, the transducer comprises a power output unit with at least one characteristic selected from: a duty cycle of about 50%, frequency of about 2 MHz, and PRF of about 20 μsec, or any combination thereof. Each possibility is a separate embodiment.

In some embodiments, the transducer is capable of a plane steering acquisition control allowing acquisition of at least nine plane waves or beams per image. Each possibility is a separate embodiment.

In some embodiments, the transducer is capable of a plane or beam steering acquisition control allowing acquisition of at least 3, at least 9, sometimes at least 18, sometimes at least 27, sometimes at least 45, or more plane waves or beams per image. Each possibility is a separate embodiment.

In some embodiments, the transducer is capable of a plane steering acquisition control allowing acquisition of between 3 and 45 plane waves per image.

In some embodiments, the transducer is capable of beam steering acquisition control allowing acquisition of at least 3, at least 9, sometimes at least 18, sometimes at least 27, sometimes at least 36, sometimes at least 45, sometimes at least 54, sometimes at least 64, sometimes at least 128, or more, beams per image. Each possibility is a separate embodiment.

In some embodiments, the transducer is capable of beam steering acquisition control allowing acquisition of between 9 and 64 beams per image, or between 9 and 128 beams per image. Each possibility is a separate embodiment.

In some embodiments, the at least nine plane waves per image comprises at least three plane angles having intervals selected from a range of intervals between about 5° and about 35°.

In some embodiments, the at least nine plane waves per image comprises at least three plane angles selected at about 5° intervals, or at about 10°, or at about 15°, or at about 20°, or at about 25°, or at about 30°, or at about 35°. Each possibility is a separate embodiment.

As another non-limiting example, the at least three plane angles may include 3 plane angles having intervals selected at about 35° intervals such as [−35°, 0°, 35°], or selected at about 20° intervals such as [−20°, 0°, 20°], or selected at about 5° intervals such as [−5°, 0°, 5°].

As another non-limiting example, the at least three plane angles may include 5 plane angles having intervals selected at about 15° intervals such as [−30°, −15°, 0°, 15°, 30°], or selected at about 10° intervals such as [−20°, −10°, 0°, 10°, 20°].

As another non-limiting example, the at least three plane angles may include 7 plane angles having intervals selected at about 10° intervals such as [−30°, −20°, −10°, 0°, 10°, 20°, 30°].

As another non-limiting example, the at least three plane angles may include 9 plane angles having intervals selected at about 5° intervals such as [−20°, −15°, −10°, −5°, 0°, 5°, 10°, 15°, 20°].

As another non-limiting example, the at least three plane angles may include 15 plane angles having intervals selected at about 5° intervals such as [−35°, −30°, −25°, −20°, −15°, −10°, −5°, 0°, 5°, 10°, 15°, 20°, 25°, 30°, 35°].

As a non-limiting example, the at least three plane angles may include intervals selected at about 5° such as [−5°, 0°, 5°] or at about 10° such as [−10°, 0°, 10°].

In some embodiments, the at least nine plane waves per image comprises at least three plane angles having intervals selected from a range of intervals at between about 5° and about 35°; and wherein each plane angle further comprises at least three angles compounded together having intervals selected from a range of intervals between about 0.1° and about 1.5°. Each possibility is a separate embodiment.

In some embodiments, each plane angle further comprises at least three angles compounded together having intervals selected from about 0.1°, or about 0.2°, about 0.5° about 1.0°, or about 1.5°. Each possibility is a separate embodiment.

In some embodiments, the at least nine plane waves per image comprises at least three plane angles selected at about 5° intervals and wherein each plane angle further comprises at least three angles compounded together having intervals selected at about 1.5° intervals or less.

As a non-limiting example, the at least nine plane waves per image comprises at least three plane angles having intervals selected at about 5° such as [−5°, 0°, 5°]; and wherein each plane angle further comprises at least three angles compounded together having intervals selected at intervals between about 1.5° such as [−6.5°, −5°, −3.5°], or about [−1.5°, 0°, 1.5°], or about [6.5°, 5°, 3.5°]. Each possibility is a separate embodiment.

As a non-limiting example, the at least nine plane waves per image comprises at least three plane angles having intervals selected at about 20° such as [−20°, 0°, 20°]; and wherein each plane angle further comprises at least three angles compounded together having intervals selected at intervals between about 0.5° such as [−20.5°, −20°, −19.5°], or about [−0.5°, 0°, 0.5°], or about [20.5°, 20°, 19.5°]. Each possibility is a separate embodiment.

The disclosed system provides a surprising advantage in the average runtime of the DSI algorithm compared to TSI.

In some embodiments, the calculation of the change in internal sound speed (m/μsec) comprises processing speed of at least about 0.3 frames per second, corresponding to at least about 2-fold increase relative to a processing speed of calculation of a corresponding change in strain (%) using Thermal Strain Imaging (TSI) (about 0.15 frames per second).

In some embodiments, the calculation of the change in internal sound speed (m/μsec) comprises processing speed of at least about 3.0 frames per second, corresponding to at least about 20-fold increase relative to a processing speed of calculation of a corresponding change in strain (%) using Thermal Strain Imaging (TSI) (about 0.15 frames per second).

In some embodiments, the DSI algorithm provides an increased speed (frame per second) in the calculation time of the change in internal sound speed of at least about 2-fold relative to a calculation time (frame per second) of a corresponding strain (%) using Thermal Strain Imaging (TSI).

In some embodiments, the DSI algorithm provides an increased speed (frame per second) in the calculation time of the change in internal sound speed of at least about 20-fold relative to a calculation time (frame per second) of a corresponding strain (%) using Thermal Strain Imaging (TSI).

In some embodiment, the DSI algorithm provides an increased speed (frame per second) in the calculation time of the change in internal sound speed of at least about 2-fold (=0.3 frames per second relative to 0.15 frames per second), or at least about 4-fold, or at least about 8-fold, or at least about 10-fold, or at least bout 12-fold, or at least about 16-fold, or at least about 20-fold (=3.0 frames per second relative to 0.15 frames per second), relative to a calculation time (frame per second) of a corresponding strain using Thermal Strain Imaging (TSI). Each possibility is a separate embodiment.

In some embodiment, the DSI algorithm provides an increased speed (frame per second) in the calculation time of the change in internal sound speed of between at least about 2-fold (about 0.3 frames per second) and about 22-fold (about 3.0 frames per second), relative to a calculation time (frame per second) of a corresponding strain using Thermal Strain Imaging (TSI) (about 0.15 frames per second).

In some embodiments, the DSI algorithm is devoid of image processing/filtering.

In some embodiments, the DSI algorithm facilitates increased plane wave sampling with respect to TSI.

In some embodiments, the treatment comprises one or more treatments selected from: high-intensity focused ultrasound (HIFU) thermal ablation therapy, cryo-ablation, and contrast agents, or any combination thereof. Each possibility is a separate embodiment.

In some embodiments, the treatment comprises high-intensity focused ultrasound (HIFU) thermal ablation therapy. In some embodiments, the treatment comprises cryo-ablation. In some embodiments, the treatment comprises use of contrast agents.

In some embodiments, the acquisition of at least two successive ultrasound (US) images is performed from a separate transducer than the HIFU transducer.

According to yet another aspect, the present disclosure provides a computer-implemented method configured to calculate a change in internal sound speed of a biological tissue indicative of a treatment, comprising: (a) receiving raw radio frequency (RF) data of at least two successively acquired ultrasound (US) images of a target area of a biological tissue; (b) applying onto the data a Dense Speed-of-Sound Shift Imaging (DSI) algorithm that:
(i) measures echo/pixel shift (μm) between the at least two successive ultrasound images; and
(ii) calculates a change in internal sound speed (m/μsec) of the biological tissue based on the echo/pixel shift; and (c) providing an output comprising feedback; the feedback comprises the calculated change in the internal sound speed of the biological tissue; and
   wherein the DSI algorithm is characterized by solving an inverse problem; and wherein the calculated change in the internal sound speed of the biological tissue comprises the solution to the inverse problem comprises.

According to some embodiments, the computer-implemented method includes iterations of the steps of the method (at least steps (a)-(c)) thereby allowing real-time non-invasive monitoring of a change in the internal sound speed of the biological tissue.

According to some embodiments, the computer-implemented method is configured to provide an output comprising feedback on the treatment; the feedback comprises a recommendation(s) at least about terminating or continuing the treatment and/or about adjusting treatment parameters. Each possibility is a separate embodiment.

According to some embodiments, the computer-implemented method is configured to provide an output comprising feedback on the treatment; the feedback comprises a recommendation(s) at least about terminating or continuing the treatment and/or about adjusting treatment parameters; and/or the calculated change in the inherent speed-of-sound of the biological tissue. Each possibility is a separate embodiment. In some embodiments, treatment parameters include but are not necessarily limited to treatment intensity and/or treatment duration. Each possibility is a separate embodiment.

In some embodiments, the computer-implemented method is configured to estimate internal temperature change of the biological tissue based on the calculated change in the internal sound speed of the biological tissue.

In some embodiments, the computer-implemented method is configured to provide an output comprising feedback on the treatment; the feedback comprises the calculated change in temperature.

The computer-implemented method encompasses the DSI algorithm, and the estimation of internal temperature change of the biological tissue based on the calculated change in the internal sound speed may be executed by the computer-implemented method, and is not necessarily part of the DSI algorithm.

The estimation of internal temperature change of the biological tissue based on the calculated change in the internal sound speed is a step the computer-implemented method is configured to execute, and is not necessarily part of the DSI algorithm.

In some embodiments, the step of estimating internal temperature change is performed by a different algorithm than the DSI algorithm (i.e., by the computer-implemented method). In some embodiments, the algorithm/computer-implemented method encompasses the DSI algorithm. In some embodiments, the algorithm/computer-implemented method encompasses the DSI algorithm and is configured to provide the temperature estimation and the feedback with recommendation(s) to a user.

In some other embodiments, the DSI can calculate the sound slowness (Equation 4) and then convert to temperature (Equation 5).

In some other embodiments, the estimation of internal temperature change of the biological tissue based on the calculated change in the internal sound speed can be performed by the DSI algorithm.

In some embodiments, the computer-implemented method is configured to provide output including temperature estimation and/or feedback with recommendation(s) to a user. Each possibility is a separate embodiment.

In some related embodiments, the estimation of internal temperature change includes converting sound slowness units to temperature by a pre-calibrated constant.

In some embodiments, sound slowness (μsec/m) is the inverse of speed sound (m/μsec) (i.e., 1/speed sound).

In some embodiments, the computer-implemented method comprises (a) receiving raw radio frequency (RF) data of at least two successively acquired ultrasound (US) images of a target area of a biological tissue.

In some embodiments, the computer-implemented method comprises (b) applying onto the data a Dense Speed-of-Sound Shift Imaging (DSI) algorithm that: (i) measures echo/pixel shift (μm) between the at least two successive ultrasound images; and (ii) calculates a change in internal sound speed (m/μsec) of the biological tissue based on the echo/pixel shift.

In some embodiments, the computer-implemented method comprises (c) providing an output comprising feedback; the feedback comprises the calculated change in the internal sound speed of the biological tissue.

In some embodiments, the computer-implemented method comprises a DSI algorithm characterized by solving an inverse problem; and wherein a solution to the inverse problem comprises the calculated change in the internal sound speed of the biological tissue. Each possibility is a separate embodiment.

According to yet another aspect, the present disclosure provides a computer-implemented method for calculating a change in internal sound speed of a biological tissue indicative of a treatment, configured to: (a) receiving raw radio frequency (RF) data of at least two successively acquired ultrasound (US) images of a target area of a biological tissue; (b) applying onto the data a Dense Speed-of-Sound Shift Imaging (DSI) algorithm that: (i) measures echo/pixel shift (μm) between the at least two successive ultrasound images; and (ii) calculates a change in internal sound speed (m/μsec) of the biological tissue based on the echo/pixel shift; and (c) estimating internal temperature change of the biological tissue based on the calculated change in the internal sound speed of the biological tissue; and (d) providing an output comprising a feedback; the feedback comprises the calculated change in the internal sound speed of the biological tissue and/or the calculated change in the internal sound speed of the biological tissue; and
wherein the DSI algorithm is characterized by solving an inverse problem; and wherein the calculated change in the internal sound speed of the biological tissue comprises the solution to the inverse problem comprises. Each possibility is a separate embodiment.

According to some embodiments, the computer-implemented method includes iterations of the steps of the method (at least steps (a)-(d)) thereby allowing real-time non-invasive monitoring of a change in the internal sound speed of the biological tissue.

According to some embodiments, the computer-implemented method is configured to provide an output comprising feedback on the treatment; the feedback comprises the calculated change in the inherent speed-of-sound of the biological tissue and/or with the calculated change in the internal temperature of the biological tissue. Each possibility is a separate embodiment.

According to some embodiments, the computer-implemented method is configured to provide an output comprising feedback on the treatment; the feedback comprises a recommendation(s) at least about terminating or continuing the treatment and/or about adjusting treatment parameters; and/or the calculated change in the inherent speed-of-sound of the biological tissue and/or the calculated change in the internal temperature of the biological tissue. Each possibility is a separate embodiment. In some embodiments, the acquisition of at least two successive ultrasound (US) images of a target area of a biological tissue is performed during a treatment performed on the tissue; thereby providing indication about the treatment. In some embodiments, the treatment can inflict a physiological change on the inherent speed-of-sound of the biological tissue and/or wherein the treatment can inflict a physiological change in the internal temperature of the biological tissue. Each possibility is a separate embodiment.

In some embodiments, the during a treatment comprises at least in parallel to the treatment, and optionally before and/or after applying the treatment on the tissue.

In some embodiments, the computer-implemented method is configured to provide feedback on the treatment; the feedback comprises an output with the calculated change in the inherent speed-of-sound of the biological tissue and/or with the calculated change in the internal temperature of the biological tissue.

The disclosed computer-implemented method provides a surprising advantage in the average runtime it takes for the DSI algorithm to calculate the change in the internal speed-of-sound.

The herein disclosed average runtime refers to the mean time it takes for each of DSI or TSI to process a frame thereby to calculate internal speed-of-sound or strain, respectively, i.e., time per frame or the inverse thereof frame per time.

In some embodiments, the calculation of the change in internal sound speed (m/μsec) comprises DSI algorithm processing speed of at least about 0.2 frames per second.

In some embodiments, the calculation of the change in internal sound speed (m/μsec) comprises DSI algorithm processing speed of at least about 0.3 frames per second.

In some embodiments, the calculation of the change in internal sound speed (m/μsec) comprises DSI algorithm processing speed of at least about 3.0 frames per second.

In some embodiments, the calculation of the change in internal sound speed comprises DSI algorithm processing speed of at least about 0.2 frames per second, at least about 0.3 frames per second, at least about 0.4 frames per second, at least about 0.5 frames per second, at least about 0.6 frames per second, at least about 0.7 frames per second, at least about 0.8 frames per second, at least about 0.9 frames per second, at least about 1.0 frames per second, at least about 1.2 frames per second, at least about 1.5 frames per second, at least about 2.0 frames per second, at least about 2.5 frames per second, at least about 3.0 frames per second, or more. Each possibility is a separate embodiment.

In some embodiments, the calculation of the change in internal sound speed comprises DSI algorithm processing speed between about 0.3 frames per second and about 3.0 frames per second.

In some embodiments, the calculation of the change in internal sound speed comprises DSI algorithm processing speed between about 0.3 frames per second and about 3.0 frames per second, corresponding to increased speed (frame per second) of calculation time of the change in internal sound speed of between at least about 2-fold (about 0.3 frames per second) and about 22-fold (about 3.0 frames per second).

In some embodiments, the DSI algorithm provides an increased speed (frame per second) in the calculation time of the change in internal sound speed (m/μsec) of at least about 2-fold relative to a calculation time (frame per second) of a corresponding strain (%) using Thermal Strain Imaging (TSI).

In some embodiments, the DSI algorithm provides an increased speed (frame per second) in the calculation time of the change in internal sound speed (m/μsec) of at least about 20-fold relative to a calculation time (frame per second) of a corresponding strain (%) using Thermal Strain Imaging (TSI).

In some embodiment, the DSI algorithm provides an increased speed (frame per second) in the calculation time of the change in internal sound speed of between at least about 2-fold (about 0.3 frames per second) and about 21.5-fold (about 3.0 frames per second), relative to a calculation time (frame per second) of a corresponding strain using Thermal Strain Imaging (TSI) (about 0.15 frames per second).

In some embodiments, the DSI is devoid of image processing/filtering; thereby allowing increasing processing speed of the calculation time of the change in internal sound speed.

In some embodiments, the DSI algorithm comprises a first step of echo/pixel shift calculation that is estimated with dense optic flow, and a second step of determining the change in internal sound speed that is calculated as the solution to the inverse problem.

In some embodiments, the DSI algorithm comprises a solution to the inverse problem; In some embodiments, the DSI algorithm comprises optical flow algorithm and a solution to the inverse problem.

In some embodiments, the Dense Speed-of-Sound Shift Imaging (DSI) algorithm uses equation 3 as a representation of equation 1. In some embodiments, Thermal Strain Imaging (TSI) uses equation 2 as a representation of equation 1. This distinguishes DSI from TSI and provides an unexpected and surprising advantage at least with respect to calculation of a change in speed sound (DSI) relative to corresponding change in thermal strain (TSI).

In some embodiments, the regularized inverse problem is represented in equation 3. In some embodiments, the inverse problem comprises regularized inverse problem.

In some embodiments, the acquisition of at least two successive ultrasound (US) images comprises at least about 20 frames per second and up to 1000 frames per second.

The disclosed method provides thermometry measurements of the biological tissue that are being executed during a treatment that is being performed on the biological tissue and deemed to inflict a change on the internal speed-of-sound and optionally also on the internal temperature of the tissue. The change in the internal temperature can be assessed by measuring the echo shift comprising a change/shift in speed-of-sound of the biological tissue.

In some embodiments, a change in internal speed-of-sound of a tissue comprises a change in internal temperature of the tissue.

Advantageously, non-invasive monitoring of a change in internal sound speed of a biological tissue in real-time enables feedback on a treatment, such as HIFU (e.g., thermal ablation) that may shift the inherent speed-of-sound of the biological tissue and may change the internal temperature of the tissue, and as a consequence may damage/destroy the tissue, for example, by inducing necrosis, cavitation and/or heat shock to the cells.

According to one aspect, the method of the present invention provides non-invasive monitoring of a change in internal sound speed of a biological tissue, comprising the steps of:
acquiring at least two successive ultrasound (US) images of a target area of a biological tissue during a treatment performed on the tissue, thereby obtaining raw radio frequency (RF) data of the acquired US images; applying onto the obtained data an algorithm that is configured to: measuring echo shift between the at least two successive ultrasound images; and calculating a change in the internal sound speed of the biological tissue based on the echo shift; and optionally, acquiring an estimation of a change in the internal temperature of the biological tissue by means of calibration of the change in the internal sound speed to a change in temperature; and wherein the algorithm comprises a Dense Speed-of-Sound Shift Imaging (DSI) algorithm. Each possibility is a separate embodiment.

As used herein, the term “acquiring at least two successive ultrasound (US) images” refers to obtaining/collecting/receiving raw radio frequency (RF) data at least of two consecutive data/time points during a treatment.

In some embodiments, at least two, at least three, at least four, at least five, at least ten, at least twenty, at least thirty, at least fourthly, at least fifty or more, consecutive data/time points are collected during a treatment. Each possibility is a different embodiment.

As used herein, the term “target area” refers to the area of the biological tissue wherein a change in the inherent speed-of-sound may occur as a consequence of the treatment which may be performed on same and/or different regions of the tissue. In some embodiments, the target area includes the focal area of the treatment and its surrounding tissue.

In some embodiments, the surrounding tissue has a radius of at least 1 mm, at least 3 mm, at least 5 mm, at least 10 mm, at least 20 mm, at least 30 mm, at least 40 mm, at least 50 mm, at least 60 mm, at least 70 mm, at least 80 mm, at least 90 mm, or at least 100 mm, from the focal area of the treatment. Each possibility is a different embodiment.

As used herein, the term “during” refers to the time period, with respect to the treatment being performed, wherein successive US images are acquired/collected and/or the raw RF data is obtained/received.

In some embodiments, successive US images are acquired and/or the raw RF data is collected before, and/or during, and/or after the treatment. In some embodiments, successive US images are acquired and/or the raw RF data is collected before the treatment begins. In some embodiments, successive US images are acquired and/or the raw RF data is collected in parallel, or at least in parallel to the treatment that is being performed. In some embodiments, successive US images are acquired and/or the raw RF data is collected after the treatment.

As used herein, the term “treatment” refers to any procedure being performed on a material, such as biological tissue, that can inflict a shift in the inherent speed-of-sound of the biological tissue which optionally/possibly may also change/shift the internal temperature of the biological tissue, possibly causing damage/destroy the tissue. In some embodiments, a treatment is a medical-related treatment. In some embodiments, a treatment is a material-engineering-related treatment.

Advantageously, the provided method and system can be used to monitor a treatment that alters the tissue speed-of-sound and optionally also the internal temperature of the tissue.

Non-limiting examples for a treatment according to the present invention include but are not limited to: high-intensity focused ultrasound (HIFU) thermal ablation therapy, cryo-ablation, adhesive bonding assessment, mechanical ablation, histotripsy, introducing a drug or other biological or chemical compound into a tissue, contrast agent imaging, or polymer congealing.

In some embodiments, the treatment comprises high-intensity focused ultrasound (HIFU) thermal ablation therapy; In some embodiments, the treatment comprises cryo-ablation; In some embodiments, the treatment comprises adhesive bonding assessment; In some embodiments, the treatment comprises mechanical ablation; In some embodiments, the treatment comprises histotripsy; In some embodiments, the treatment comprises introducing a drug or other biological or chemical compound into a tissue; In some embodiments, the treatment comprises polymer congealing; In some embodiments, the treatment comprises contrast agents imaging; In some embodiments, the treatment comprises contrast agents imaging.

In some embodiments, thermal ablation treatment comprises laser treatment. In some embodiments, thermal ablation treatment comprises tumor ablation. In some embodiments, thermal ablation treatment comprises contrast agents.

Non-limiting examples of thermal ablation treatments using laser include skin resurfacing, cavity preparation, biopsies, lesion removal and/or tumor ablation.

In some embodiments, thermal ablation treatment comprises skin resurfacing, cavity preparation, biopsies, lesion removal, and/or tumor ablation. In some embodiments, the tumor is benign or malignant.

In some embodiments, the treatment inflicts a change on the inherent speed-of-sound of the biological tissue. In some embodiments, the treatment inflicts a change in the internal temperature of the tissue. In some embodiments, the treatment inflicts a change on the inherent speed-of-sound of the biological tissue and optionally also on the internal temperature of the tissue.

In some embodiments, the treatment comprises mechanical procedure, non-mechanical procedure and/or non-invasive procedure.

In some embodiments, the treatment damages/destroys the tissue. In some embodiments, the treatment induces necrosis of the biological tissue. In some embodiments, the treatment induces cavitation and/or heat shock to the cells.

In some embodiments, the treatment induces change in sound speed by introducing a drug or other biological compound into the tissue.

As used herein, the term “algorithm” may refer to a computer-implemented method comprising of a Dense speed-of-sound shift Imaging (DSI) estimating algorithm that provides solutions to the round-trip problem/echo shift problem thereby accurately measuring the echo/pixel shift and calculating the change in the internal sound speed of the biological tissue based on the echo/pixel shift, or in some embodiments, the term “algorithm” may refer to the DSI algorithm itself.

In some embodiments, the algorithm may further provide an estimation of a change in the internal temperature of the biological tissue by means of calibration of the change in the internal sound speed to a change in temperature.

In some embodiments, echo shift is calculated using the Horn-Shunck or Farneback Dense optical flow algorithm. In some embodiments, the dense optical flow algorithm incorporates a spatial smoothness prior, forcing the resulting echo shifts to be spatially coherent as expected.

In some embodiments, the algorithm comprises a Dense Speed-of-Sound Shift Imaging (DSI) algorithm.

In some embodiments, optical flow algorithms utilize pyramid calculation schemes to incorporate both low and high resolution information quickly. Thus, advantageously, optic flow provides a faster, more spatially relevant solution for echo shift data. In some embodiments, the algorithm comprises Gunnar Farneback's method, based on polynomial expansion.

In some embodiments, echo shift is calculated with other dense algorithms like homography.

In some embodiments, echo shift is calculated by means of non-dense algorithms like cross-correlation or phase analysis. Although this does not provide a coherent input to the next step, the inverse problem solution will still force the result to be spatially coherent as expected.

In some embodiments, echo shift measurements, are used to calculate the sound speed change. In thermal strain imaging (TSI), this is done by differentiating the echo shift in the axial direction to produce thermal strain, which can be directly calibrated to the temperature shift. However, this ignores the spatial coherence of the treatment, such as in the case of HIFU-based therapy. Advantageously, instead, the echo shift from several plane waves can be used to predict the change in speed-of-sound using an inverse problem method regularized with a spatial smoothness prior, and the result can be calibrated to yield a similar temperature shift.

Therefore, the algorithm combines the optical flow algorithm and inverse problem optimization to create a sound speed shift estimating algorithm that can incorporate a wide array of information while forcing both the input and output to obey spatial regularization constraints.

In some embodiments, the algorithm comprises a Dense Speed-of-Sound Shift Imaging (DSI) algorithm.

In some embodiments, the Dense Speed-of-Sound Shift Imaging (DSI) algorithm comprises a first step of translation estimation (i.e., calculation of Δd) that is estimated with dense optic flow and a second step of calculating the slowness deviation (Δv) that is calculated as the solution to the regularized inverse problem

As used herein, the term “translation” refers to Δd.

As used herein, the term “slowness deviation” refers to Δv.

### Theory:

In the following section, the mathematical relationship between the predicted echo shift or change in round-trip time δτ and the temperature change δT along the wave propagation path is described. It was previously noted that the relationship of this echo shift to temperature and local sound speed is given by the following equation:

$${δ}_{τ} = 2 ∮ \frac{1 + β ( z ) δ T}{c ( z , T )} - \frac{1}{c ( z , {T}_{0} )} dz , \qquad ( 1 )$$

where the difference in round-trip time or is the cumulative sum of the inverse of changes in local sound speed c(z,T) due to a temperature change δT from the initial temperature T₀ in tissue with a thermal expansion coefficient of β(z) along the wave propagation path.

In thermal strain imaging, this integral is differentiated along the propagation path and several assumptions can be made to simplify the equation for calculation of temperature change from echo latency:

$$\frac{\partial }{\partial z} ( {δ}_{τ} ) = {α}_{1} \times δ T , \qquad ( 2 )$$

where α1 is a medium-specific parameter describing the thermal expansion coefficient and linear relationship of sound speed with temperature, and can also be thought of as a unit conversion parameter from thermal strain to temperature. A method for ultrasonic estimation of temperature change from thermal strain based on equation (2) starts by calibration of the constant α1 using a known ground truth temperature sensor. Here, a linear relation between the speed-of-sound and the change in temperature is assumed because the interest in this disclosure is in improving the estimation of sound speed shift, which is the underlying cause of thermal strain, rather than the calibration to temperature measurements. For water-based soft tissue, the constant α1 has been reported to be −0.1%° C.⁻¹ change in thermal strain for a 1° C. increase in temperature.

In the disclosed method, DSI, a new and advantageous approach for solving equation (1) is suggested. Rather than differentiate the line integral as is common in thermal strain imaging, in some embodiments, the problem is assumed to be linearizable. Therefore, equation (1) can be vectorized. The integral is represented as a matrix, denoted M below and the following equation is obtained:

$$Δ \underline{d} = M Δ \underline{v} \qquad ( 3 )$$

where the pixel shift Δd is observed in a plane wave B-mode image due to an echo shift of δτ. This Δd is the result of integrating the integrand Δv, which represents the change in sound slowness according to the following equation:

$$Δ \underline{v} = \frac{1 + β ( z ) δ T}{c ( z , T )} - \frac{1}{c ( z , {T}_{0} )} . \qquad ( 4 )$$

Once the integral operand Δv is isolated, it is equivalent to the derivative of (1) such that equation (2) can be rewritten without the derivative to obtain:

$$Δ \underline{v} = {α}_{2} \times δ T \qquad ( 5 )$$

with α2 assuming the tissue-dependent role of units conversion previously denoted in equation (2) as α1. In temperatures up to 40° C., the relationship between sound speed and temperature change is mostly linear and α2 should correspond to a change of up to 1 ms⁻¹ for a 1° C. rise in temperature, depending on the target tissue.

In practice, the inventors wanted to measure the pixel shift, calculate Δv and estimate the underlying temperature change, so the following inverse problem is formulated:

$${M}^{- 1} Δ \underline{d} = Δ \underline{v} . \qquad ( 6 )$$

This problem is over-defined since many Δd images can be acquired from a particular field of view representing a single Δv. After transmitting multiple angled plane waves into the target tissue, a Tikhonov pseudo-inverse was utilized to calculate the optimal Δv satisfying the data. While this increases the potential for a reliable result, in some embodiments, it further incorporate a smoothness prior to ensure spatial coherence. In some embodiments, the spatial gradient was regularize in the axial, lateral, and diagonal directions using L2 regularization. Thus, the detected change in sound slowness Δv is estimated by solving the following least squares problem:

$$Δ \underline{\hat{v}} = arg {min}_{Δ \underline{\underline{v}}} {\vert Δ d - M Δ \underline{v} \vert}^{2} + Γ \vert ∇ {( Δ \underline{v} )}^{2} . \qquad ( 7 )$$

The pipeline of the DSI method utilizes the acquisition of ultrasound plane wave B-Mode images. The pixel shift at consecutive time intervals for a particular steering angle is computed via optical flow to produce the measured Δd vector. This vector is inverted as an inverse problem to receive Δv. Finally, the units of Δv are converted to temperature by a pre-calibrated constant.

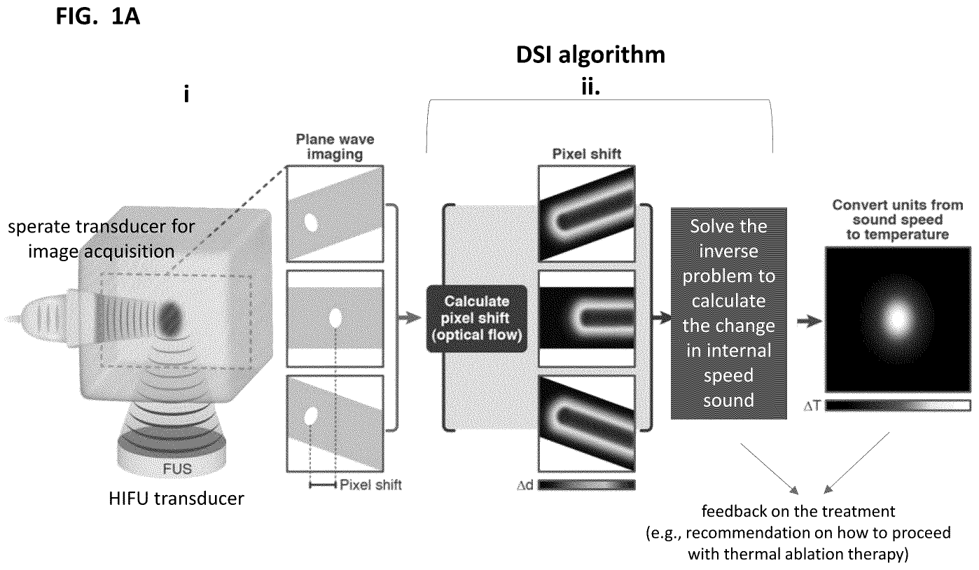

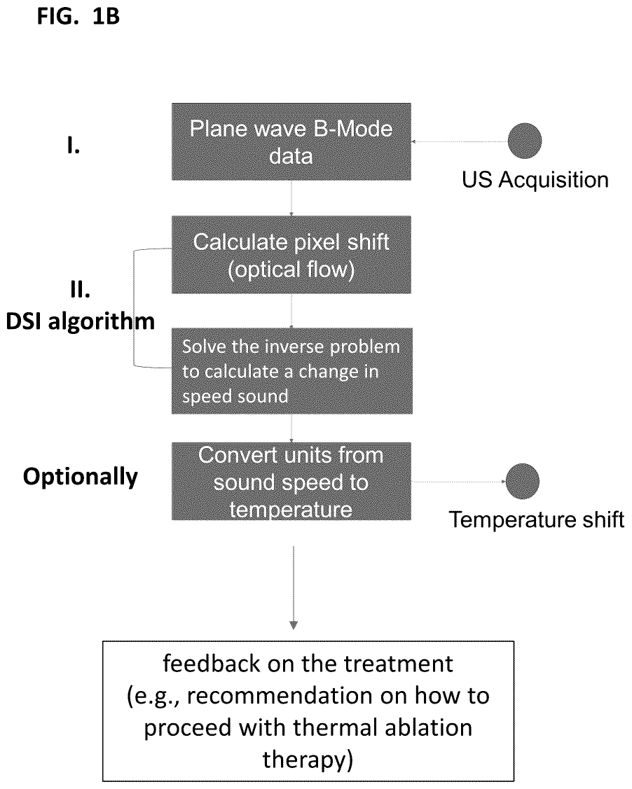

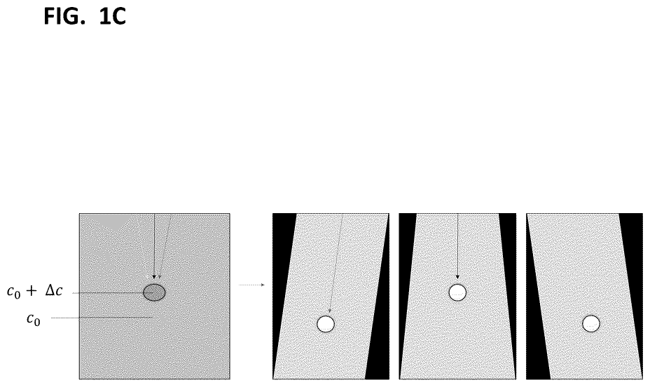

Reference is now made to FIG. 1 schematically illustrating the steps of the method for non-invasive monitoring of a change in a temperature.

In some embodiments, the method further comprises an iteration of the steps of the method that allows real-time non-invasive monitoring of internal sound speed shift of a biological tissue and feedback on the treatment.

As used herein, the term “feedback” refers to a decision being made regarding the continuation, termination or adjustment of the treatment based on the calculated change in the sound speed of the target area of the biological tissue, and provided as a recommendation to the user. The decision being made regarding the continuation, termination or adjustment of the treatment based on the calculated change in the sound speed of the target area of the biological tissue.

In some embodiments, the feedback may include vocal, visual or textual presentation of the recommendation. In some embodiments, the feedback may also include vocal, visual or textual presentation of the calculated change in internal speed sound of the tissue and/or internal temperature of the tissue.

In some embodiments, the feedback may be part of an output of the computer-implemented method and may be presented by an output device such as a monitor. In some embodiments, the part of the computer-implemented method that makes the decision about a recommendation to the user may be a machine learning algorithm. In some embodiments, the computer-implemented method comprises a machine learning algorithm.

In some embodiments, the algorithm provides a recommendation of a decision to be made.

In some embodiments, the provided recommendation includes a recommendation to continue, to stop, or to adjust parameters of the treatment that affect the change in sound speed of the biological tissue. Each possibility is a separate embodiment.

In some embodiments, the acquisition of at least two successive ultrasound (US) images comprises acquiring plane waves B-mode data.

In some embodiments, the acquisition of at least two successive ultrasound (US) images comprises acquiring at least 1, at least 3, at least 6, at least 9, at least 12, at least 15, at least 18 or more, plane waves or beams per imaging sequence. Each possibility is a separate embodiment.

In some embodiments, the acquisition of at least two successive ultrasound (US) images comprises acquiring at least 9 plane waves or beams per imaging sequence. Each possibility is a separate embodiment.

In some embodiments, the acquisition of at least two successive ultrasound (US) images comprises acquiring at least 1 plane waves or beams per imaging sequence. Each possibility is a separate embodiment.

In some embodiments, the acquisition of at least two successive ultrasound (US) images comprises acquiring one or more ultrasound beams per imaging sequence.

As used herein, the term “imaging sequence” refers to the acquired US images and/or the collected raw RF data from which the internal sound speed and optionally the internal temperature of the biological tissue at a specific timepoint during the treatment can be calculated based on the echo shift. The change in sound speed is calculated between two imaging sequences. The term imaging sequence is interchangeable with the term “image” or “frame”.

In some embodiments, an imaging sequence comprises acquiring at least 9 plane waves.

In some embodiments, an imaging sequence comprises acquiring at least 1, sometimes at least 3, sometimes at least 6, sometimes at least 9, sometimes at least 12, sometimes at least 15, or sometimes at least 18 plane waves or beams. Each possibility is a separate embodiment.

In some embodiments, an imaging sequence comprises acquiring between 1 to 18, sometimes between 1 to 15, or sometimes between 1 to 12 plane waves. Each possibility is a separate embodiment.

In some embodiments, the at least 9 plane waves per imaging sequence comprises at least three plane angles of about 5° intervals, for example [−5°, 0°, 5°], and wherein each plane angle further comprises at least three angles selected at about 1.5° intervals, for example [−6.5°, −5°, −3.5°].

In some embodiments, the three plane angles comprise about 5° intervals. In some embodiments, the at least three angles selected at about 1.5° intervals. In some embodiments, the echo shift comprises a change/shift in speed-of-sound of the biological tissue.

In some embodiments, the treatment can inflict a change on the inherent speed-of-sound of the biological tissue. In some embodiments, the treatment can inflict a change in the internal temperature of the tissue.

In some embodiments, the treatment comprises mechanical procedure, non-mechanical procedure and/or non-invasive procedure.

In some embodiments, the treatment comprises high-intensity focused ultrasound (HIFU) thermal ablation therapy. In some embodiments, the thermal ablation therapy comprises tumor ablation. In some embodiments, the thermal ablation therapy comprises contrast agents. In some embodiments, contrast agents may include such agents as, but not limited to: nanobubbles, nanodroplets, microbubbles, gas vesicles or combinations thereof.

In some embodiments, the treatment comprises high-intensity focused ultrasound (HIFU) thermal ablation therapy, cryo-ablation, adhesive bonding assessment, mechanical ablation, histotripsy, introducing a drug or other biological or chemical compound into a tissue, contrast agent imaging, or polymer congealing.

In some embodiments, non-invasive monitoring of a change in speed-of-sound of a biological tissue is performed in-vivo or ex-vivo. In some embodiments, non-invasive monitoring of a change in internal temperature of a biological tissue is performed in-vivo or ex-vivo; In some embodiments, the biological tissue is in-vivo or ex-vivo.

In some embodiments, the biological tissue is cancerous; In some embodiments, the biological tissue is a tumor.

In some embodiments, the biological tissue comprises a soft tissue, solid tissue, bone, fat, cartilage or chemical deposit, lymphatic vessel or a blood vessel. Each possibility is a separate embodiment.

In some embodiments, the acquisition of the at least two successive ultrasound (US) images during the treatment is performed before, in parallel and/or after the treatment. Each possibility is a separate embodiment.

In some embodiments, the acquisition of the at least two successive ultrasound (US) images during the treatment is performed in parallel, or at least in parallel to the treatment. Each possibility is a separate embodiment.

In some embodiments, the acquisition of the at least two successive ultrasound (US) images is performed from a separate transducer than the HIFU transducer.

In some embodiments, the imaging transducer and the HIFU transducer are the same unit having different roles at different time points with respect of the treatment.

According to another aspect, the system of the present invention provides non-invasive monitoring of a change in internal sound speed of a biological tissue, comprising:
at least a single transducer capable of acquiring at least two successive ultrasound (US) images of a target area during a treatment performed on the tissue; a transmitter capable of beamforming obtained raw radio frequency (RF) data of the acquired US images; a processing unit capable of receiving the RF data and executing thereon an algorithm that measures echo shift between two successive ultrasound images and calculate a change in internal sound speed based on the echo shift; and optionally, calibrating the change in internal sound speed to provide a change in internal temperature; and a monitor capable of presenting the change in the sound speed/temperature; and wherein the algorithm comprises or essentially consists of a Dense Speed-of-Sound Shift Imaging (DSI) algorithm. Each possibility is a separate embodiment.

In some embodiments, the transducer comprises a power output unit with a duty cycle of about 50%, frequency of about 2 MHz, and PRF of about 20 μsec.

In some embodiments, the transducer is capable of a plane or beam steering acquisition control allowing acquisition of at least 1, sometimes at least 9, sometimes at least 18 plane waves or beams per imaging sequence. Each possibility is a separate embodiment.

In some embodiments, the at least nine plane waves per image comprises at least three plane angles of about 5° intervals, for example [−5°, 0°, 5°], and wherein each plane angle further comprises at least three angles selected at about 1.5° intervals, for example [−6.5°, −5°, −3.5°].

In some embodiments, the treatment comprises high-intensity focused ultrasound (HIFU). In some embodiments, the HIFU is a thermal ablation therapy.

In some embodiments, the acquisition of the at least two successive ultrasound (US) images is performed from a separate transducer than the HIFU transducer.

According to yet another aspect, there is provided a computer-implemented method for calculating a change in internal speed-of-sound of a biological tissue, comprising the steps of: receiving raw radio frequency (RF) data of at least two successive acquired ultrasound (US) images of a target area; applying onto the received data an algorithm that is configured to: measures echo shift between two successive ultrasound images; and calculating a change in sound speed based on the echo shift; and optionally calibrating the change in internal sound speed to provide a change in internal temperature; and providing an output comprising the change in the internal temperature; and wherein the algorithm comprises a Dense Speed-of-Sound Shift Imaging (DSI) algorithm. Each possibility is a separate embodiment.

In some embodiments, the raw radio frequency (RF) data of at least two successive acquired ultrasound (US) images comprises plane waves B-mode data.

In some embodiments, the raw radio frequency (RF) data of at least two successive acquired ultrasound (US) images comprises data of imaging of at least nine plane waves per image.

In some embodiments, the at least nine plane waves per image comprises at least three plane angles of about 5° intervals, for example [−5°, 0°, 5°], and wherein each plane angle further comprises at least three angles selected at about 1.5° intervals, for example [−6.5°, −5°, −3.5°].

In some embodiments, Dense Speed-of-Sound Shift Imaging (DSI) algorithm comprises a first step of translation calculation that is estimated with dense optic flow, and a second step of calculating the slowness deviation that is calculated as the solution to the regularized inverse problem.

In some embodiments, Dense Speed-of-Sound Shift Imaging (DSI) algorithm comprises optical flow algorithm, spatial regularization constraints, and an inverse problem optimization.

According to some aspects, there is provided a method of non-invasive monitoring of a change in internal sound speed of a material, comprising the steps of: acquiring at least two successive ultrasound (US) images of a target area of a material during a treatment performed on the material, thereby obtaining raw radio frequency (RF) data of the acquired US images; applying onto the obtained data an algorithm that is configured to measure echo shift between the at least two successive ultrasound images; and calculating a change in the internal sound speed of the material based on the echo shift; and optionally, acquiring an estimation of a change in the internal temperature of the material by means of calibration of the change in the internal sound speed to a change in temperature; and wherein the algorithm comprises or essentially consists of a Dense Speed-of-Sound Shift Imaging (DSI) algorithm. Each possibility is a separate embodiment.

In some embodiments, the material comprises a chemical material, a biological material, or a combination thereof. In some embodiments, the biological material is a biological tissue. In some embodiments, the chemical material is a drug or chemical compound. In some embodiments, the material comprises a drug or other biological or chemical compound, or combination thereof. In some embodiments, the treatment is polymer congealing.

In some embodiments, any treatment that changes the speed of sound in a specific material (i.e., biological or chemical, or combination thereof) can be monitored using the methods and system of the present disclosure, including medical-related treatments and material engineering-related treatments, such as polymer congelation.

According to one aspect, the present disclosure provides a method for real-time non-invasive imaging and monitoring of a local speed-of-sound shift in a biological tissue with a dense algorithm, for the purpose of temperature estimation.

Advantageously, in some embodiments, the DSI method improves the calculation time and spatial coherence of the result, relative to those achieved using TSI method.

According to some embodiments the proposed DSI algorithm is robust and could be used to monitor additional applications that induce a speed-of-sound shift, including but not-limited to one or more applications selected from: high-intensity focused ultrasound (HIFU) thermal ablation therapy, cryo-ablation, adhesive bonding assessment, mechanical ablation, histotripsy, introducing a drug or other biological or chemical compound into the tissue, contrast agents, and polymer congealing, or any combination thereof. Each possibility is a separate embodiment.

The common method for ultrasonic thermometry is TSI which is based on a cross-correlation search and local axial gradient. The present disclosure provides several surprising advantages for the DSI algorithm compared to TSI, in guidance/monitoring of changes in speed-of-sound at least during HIFU, cryoablation and contrast agents. First, across all experiments, it is clear that the smoothness priors used in the optical flow calculation and inverse problem regularization greatly improve the spatial coherence of the results.

In some embodiments, the method comprising DSI is characterized by providing improved spatial coherence compared to TSI. In some embodiments, spatial coherence comprises optical flow calculation and/or inverse problem regularization.

Reference is now made to FIGS. 3A-3C (i-iv), where the image processing steps that work well in the simulated TSI results do not generalize well to ex vivo, and the TSI pixel shift estimation gives a large focal area. Heavy smoothing was necessary to reach an adequately coherent TSI image using Savitzky-Golay filtering.

A second surprising advantage of DSI is not needing the image processing/filtering. In TSI, the results are quite noisy (FIG. 3C ii), so image processing is used to make them look nicer. The result of FIG. 3C (ii) already includes this filtering and still looks noisy, with respect to DSI (FIG. 3C iv). It is an undesirable result which also requires processing time. Advantageously, DSI doesn't require any such filtering/image processing.

In some embodiments, the DSI is devoid of image processing or filtering with respect to TSI; thereby allowing increasing processing speed of the calculation time of the change in internal sound speed.

In the future, knowledge of the shape of a HIFU beam in the transverse imaging plane can intuitively incorporate further a priori information into the DSI inverse problem solution to produce a more complex regularization scheme. This improvement can be further capitalized on by increasing the amount of plane waves to more heavily oversample the sound slowness field. Both of these concepts can be used to reduce the blurring (increase coherence) caused by trivial L2 spatial regularization (FIG. 2C) as depict in Equation 7.

Reference is now made to FIGS. 2B-C demonstrating improvement in L2 spatial regularization.

Yet another, third surprising advantage of DSI is its improved runtime. TSI and other correlation-based tracking methods are insufficient for real-time use when the region of interest is the entire frame. In this case it is preferable to make use of dense algorithms incorporating all of the available information simultaneously, while eliminating the need for image processing.

In some embodiments, the method comprising DSI is devoid of the need for image processing; thereby improving the runtime with respect to TSI.

In some embodiments, removal of the search step of TSI and replacement with optical flow provided a large part of the performance boost, but DSI's slowness estimation is also very fast due to the use of largely sparse matrices in solving the inverse problem. In ex vivo samples a slowness deviation was detected in order of 3 μs m⁻¹, which corresponds to a sound speed change of 7 m s⁻¹ from a base sound speed of 1540 m s⁻¹. In some embodiments, this translates to a difference in speed-of-sound of 0.45% between the TSI and DSI methods. The temperature estimation results were similar between the ground truth thermocouple measurements, the TSI and DSI methods.

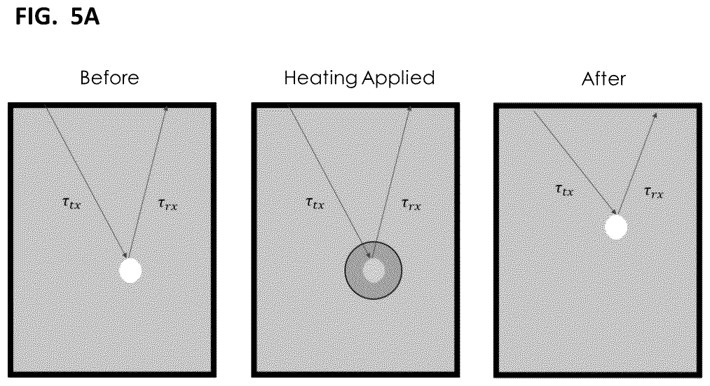

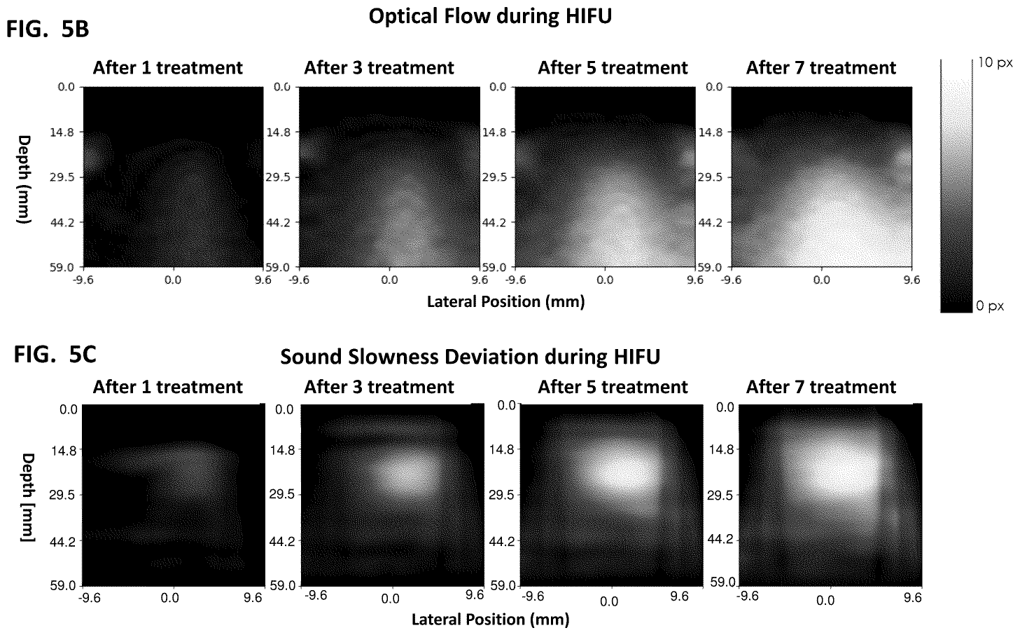

Reference is now made to FIGS. 5B-5C demonstrating optical flow and slowness estimation measurements during HIFU.

Several considerations should be made when implementing the DSI algorithm. First, there are inherent tradeoffs in selecting the steering angles and regularization constants described in this algorithm. Selecting the sub-angles used for coherent compounding of the main angles is a direct tradeoff between SNR and resolution with frame rate, as in any coherent compounding scheme. The main angles play a much more prominent role in both processing time and resolution. Pixel tracking is performed on each angle individually, and the inverse problem calculation incorporates data from each of the main angles simultaneously, so the complexity of digital processing done in this study is linear with respect to the number of main steering angles. The scanning step of the plane waves must be selected so that high SNR is achieved by the sub-angles while adequately sampling the viewing field, with the goal to maximize the number of pixels in the region of interest being sampled by more than one plane wave. It is the cross-information acquired by separate plane waves on each pixel that improves the accuracy of the inverse problem solution. In addition, the RF sampling parameters must be fine-tuned for imaging during therapy. The most important consideration is the ultrasonic frequency: using a high frequency improves the resolution which is helpful for pixel tracking, but high frequency ultrasound comes with several fallbacks. Increased tissue absorption associated with high frequencies causes the SNR to drop significantly at depth, and can also increase the thermal dose when used in between consecutive HIFU pulses. More expensive hardware is required to support the RF sampling rate required for such frequencies as well. Still, the ultrasound frequency should be maximized to improve imaging with these considerations in mind.

The DSI algorithm provides reasonable, robust slowness deviation estimation in real-time applications. In some embodiments, the DSI algorithm is implemented in real-time as shown by the performance benchmark (Example 6), by storing the pseudo-inverse matrix, beamforming each plane wave angle independently, tracking the pixel motion, and solving the inverse problem with a simple matrix multiplication.

In some embodiments, the speed up in the algorithm run time could facilitate its implementation as part of a closed-loop HIFU controller, or in other embodiments, it can be used with matrix array transducers to enable real-time 3D thermometry.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Material and Method

Ultrasound Setup—HIFU was performed with a 2 MHz spherically focused single element transducer (H107, Sonic Concepts, Bothell, WA, USA) that was controlled by a transducer power output unit (TPO-200, Sonic Concepts, Bothell, WA, USA). This transducer has a diameter of 64 mm and is focused at a depth of 45 mm. Each transmitted pulse consisted of a sinusoid with a peak negative pressure of 3.25 MPa and a duty cycle of 50%. These parameters matched standard hyperthermia experiments. To create a steady increase in temperature, the therapeutic transducer transmitted with a pulse repetition time of 20 us with bursts that consisted of 20 cycles. HIFU treatment was performed in intervals of 45 s lasting a total duration of 270 s. After every interval, imaging was performed, and the results stored before the next HIFU cycle began.

The therapeutic transducer was placed face-up at the bottom of a custom water tank. A holder was located at the focal spot, where the sample was placed. Imaging was performed using an L12-5 50 mm linear transducer (ATL Philips, WA, USA) placed perpendicular to the sample and coupled with ultrasound gel (FIG. 3A). A programmable ultrasound system (Vantage 256, Verasonics Inc., WA, USA) was used for imaging of a 59 mm×40 mm field of view. A plane steering acquisition protocol was used to acquire nine plane waves per imaging sequence. Three main steering angles were selected at 5° intervals [−5°, 0°, 5°], each composed of three steering angles selected at 1.5° intervals (for example [−6.5°, −5°, −3.5°]). Raw RF data was stored for offline processing.

Two types of samples were used. The first was a tissue mimicking phantom. The phantom was made by heating a combined mixture of 1% agarose powder (A10752, Alfa Acsar, MA, USA) with deionized water until boiling. 1% Silicon Carbide (57391, Sigma Aldrich, MO, USA) was added to the cooling mixture as acoustic scatterers, and the result was poured into a mold to congeal. Next, fresh ex vivo chicken breast samples were used. Each chicken breast sample was cut from the thickest part of a chicken breast in a single piece that could fill the entire field-of-view (pieces with a size of 59 mm×40 mm×15 mm3). The samples were placed in a 3D printed cartridge at the focal spot of the transducer. To minimize reflections 6 mm rubber was placed around the sample and coupled with ultrasound gel. As a ground truth reference, a thermocouple was placed near the focal spot. The probe was operated from MATLAB during the imaging sequence through an Arduino Uno R3.

The pressure amplitude in this setup was calibrated using a needle hydrophone (NH0500, Precision Acoustics, Dorchester, UK) with an active aperture of 0.5 mm connected to an oscilloscope (MDO3024, Tektronix, OR, USA). The hydrophone was placed at the focal spot of the therapeutic transducer, such that the measured amplitude was maximal in the x, y, and z directions.

Post-Processing and Temperature Change Calculation—RF data was beamformed and interpolated onto a 599×390 pixel² grid, such that the pixel size was 0.01 mm in both the axial z direction as well as the lateral x direction. Each image was then Hilbert transformed, and the 1.5° angled plane waves sub-sets were compounded such that each image acquisition provided three main steering angles. For example, the set [−6.5°, −5°, −3.5°] was compounded to yield a single image of −5°. This unique compounding has been used previously to enhance the signal-to-noise ratio in preparation for the pixel-tracking step. The dense optic flow was calculated between consecutive images at each of the main steering angles using Farneback's method. The three calculated pixel-shift images acquired at each time interval were down-sampled ×5 in the lateral direction and ×10 in the axial direction, then stacked to create the data vector. The matrix M described in equations (6) and (7) was pre-calculated and stored as a sparse matrix. Given a new data vector Δd, a simple matrix multiplication was performed to receive the slowness deviation. The regularization parameters, which are responsible for the smoothness of the result, were selected empirically to give the best possible resulting image. They can be fine-tuned based on the size and smoothness desired for an application of the algorithm. In some embodiments, the use of regularization parameters Γx=10, Γz=Γxz=1 was chosen. This means a certain level of smoothness was required for each of a pixel's 8 neighbors independently: Γx controls the smoothness with regard to the left-right neighbors, Γz controls the smoothness with respect to the pixel neighbors above and below the pixel, and Γxz controls the diagonal smoothness. The resulting image of pixel change in sound slowness was up-sampled back to the original image dimensions, then summed and compared to the expected temperature change to fit a linear curve, producing the calibration constant α2.

As a reference to DSI, TSI was also computed using the same data sets. Implementation included a ID cross correlation search algorithm along each plane wave's propagation axis. The pixel shift was smoothed in the lateral direction with a Savitzky-Golay filter of length 13.4 mm and then the axial gradient was estimated with a second, gradient estimating Savitzky-Golay filter of length 15.6 mm. Each of the main angles described previously was analyzed separately, then the results were compounded for each time step.

Both algorithms were implemented in python. DSI's optical flow component made use of the OpenCV library, while the inverse problem solution was implemented as a sparse matrix multiplication using scipy. TSI, which requires a complex cross-correlation search, was implemented in numba as just-in-time compiled python code utilizing parallel processing to improve performance.

### Example 1—Overview of a Method for Non-Invasive Monitoring of a Change in a Temperature

The disclosed method includes acquiring successive ultrasound (US) images of the ROI of the tissue during the medical procedure (e.g., thermal ablation) and obtaining raw radio frequency (RF) data of the plane wave B-mode as illustrated in FIG. 1A (i) and FIG. 1B (i).

Since during thermal ablation, the inherent sound speed of the tissue is altered, the tissue is perceived to “move” along the axial direction of the ultrasound beam as illustrated in FIG. 1C. The figure illustrates a homogenous tissue with an area of sound speed varying from the background due to an “object”/treatment, could be a thermal ablation or a pocket of microbubbles MBs), and further illustrates 3 plane wave angles (−5, 0, 5, for example) being transmitted into the tissue, each plane wave perceives the object as being slightly displaced due to the effect of equation 1.

Accordingly, a Dense Speed-of-Sound Shift Imaging (DSI) algorithm is applied on the data, as illustrated in FIG. 1A (ii) and FIG. 1B (ii). First, the translation Δd is estimated with dense optic flow (pixel shift). Next, the slowness deviation Δv (change in speed sound) is calculated as the solution to the regularized inverse problem.

This process is exemplified in Example 2 and Example 3, and includes calculating the translation of each pixel, thereby allowing the shift in sound speed to be reconstructed, and the slowness deviation to be calculated.

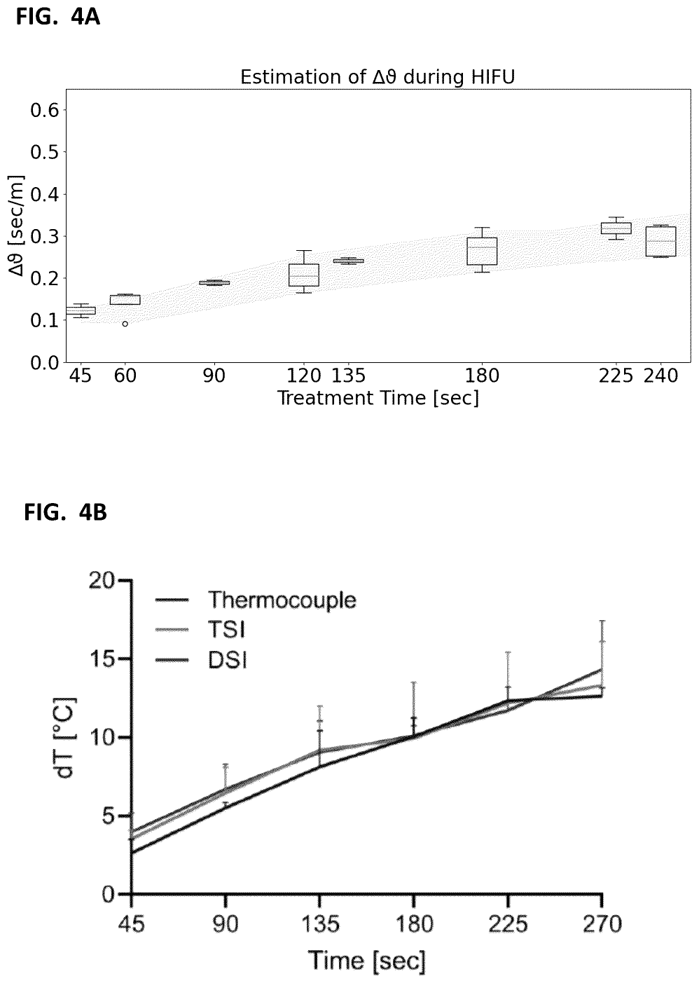

Finally, the result is used to estimate the temperature change by multiplying it by a scalar converting units to temperature shift, as illustrated in FIG. 1A (iii) and FIG. 1B (iii), and exemplified in Example 4 and FIG. 4B.

The theory underlying the DSI algorithm is presented in Equations (1) to (7), described elsewhere hereinabove in the disclosure particularly, in equations 3-4, and also in equations 6-7.

### Example 2—Non-Invasive Monitoring of a Change in a Temperature, Performed on Agarose Tissue-Mimicking Phantom

Briefly, plane wave B-Mode data was acquired using successive ultrasound (US) images as described hereinabove. To validate the algorithm, RF data from an agarose tissue-mimicking phantom was collected. To simulate a shift in speed of sound, a circular region was selected and pixels along each ultrasound acquisition were translated along the axial direction. (FIGS. 2A-2C).

Advantageously, the resulting estimated sound speed deviation has better spatial coherence, resolution, and contrast than the current standard TSI. In addition, the calculation is much faster and is optimized for real-time imaging.

In more detail, to validate the method, a simulated pixel-shift was induced in a set of steered plane wave images acquired from agarose tissue-mimicking phantom by manually introducing a pixel shift into the RF data captured from real images, implemented by translating pixels along each plane wave's propagation axis. A circular lesion mimicking a heated area was simulated within the images by introducing a virtual temperature shift of approximately 10° C., at a circular area centered in the image with a radius of 5 mm (FIG. 2A). This virtual heating was entirely controlled so that the algorithms could be compared to a known reference that was not affected by environmental factors like density, imaging parameters, or transducer alignment. In addition, agarose is less absorbent of ultrasonic energy than soft tissue, and in some embodiments, a virtual heat source representative of soft tissue was introduced.

The ground truth model is presented alongside TSI thermal strain calculation and DSI slowness deviation reconstruction (FIG. 2A right panel; marked (ii), (iv), and (vi), respectively). The pixel shift of the TSI method provides a lateral full-width at half-max (FWHM) of 8.19 mm, while the DSI method gives a FWHM of 8.79 mm. The ground truth lateral FWHM is 8.49 mm (FIG. 2A left panel (i), (iii), (v)).

In terms of lesion dimensions, the lateral FWHM in TSI reconstruction is 10.56 mm while DSI achieves 9.57 mm (FIG. 2B). The ground truth was 9.77 mm in each direction. In the axial direction, TSI gives a FWHM of 7.89 mm while DSI gives 15.5 mm (FIG. 2C).

Overall, there is a smoother improved result with the DSI method, relative to Ground truth and/or TSI methods, characterized by reconstructed speed-of-sound deviation having enhanced spatial coherence with: (i) a pixel shift providing an increase in lateral full-width at half-max (lateral FWHM) of between about 3% to about 7%; and (ii) lesion dimensions providing a decrease in lateral FWHM of at least about 10% and an increase in axial FWHM of about 2-fold.

### Example 3—Non-Invasive Monitoring of a Change in a Temperature, Performed on Ex-Vivo Chicken Breast During a Thermal Ablation

Next, ex-vivo chicken breast experiments were conducted. The DSI algorithm was evaluated on plane wave B-Mode data that was acquired using successive ultrasound (US) images of ex-vivo chicken breast during a thermal ablation induced by a 2 MHz HIFU transducer. (FIG. 3A-3D)

Advantageously, the result shows that the disclosed method comprising DSI algorithms provides estimation of the sound slowness deviation. An echo shift with intuitive coherence is generated and the resulting focal point of the slowness deviation in (FIG. 3C-3D) has similar radius to the focal spot apparent on the sample tissue. (FIG. 3B).

In more detail, HIFU was applied at the center of the sample, and an ablated region was visible at the center of the images of the sliced sample FIG. 3B. A thermocouple was located at the focal spot to serve as the ground truth measurement. The imaging array acquired plane waves on the transversal plane of the sample. The recorded pixel shift and the reconstructed image were compared between the TSI and DSI methods (FIG. 3C i-iv), and the focal area was defined as the number of pixels above half the maximal sound slowness deviation multiplied by the resolution in each axis. The lesion detected with TSI has a focal area of 226 mm² (FIG. 3C ii) while the DSI lesion recorded a focal area of 177 mm² (FIG. 3C iv), a reasonably similar result.

Next, the effect of the HIFU duration on the DSI algorithm performance was assessed (FIG. 3D i-vi). Larger speed of sound shift was observed with an increase in treatment duration. The detected slowness deviation and focal area for each time step in FIG. 3D are displayed in Table 1 below.

TABLE 1
Detected slowness deviation and focal area
of the treatment spot based on FIG. 3D.

| Treatment | Max slowness | Measured focal |
|---|---|---|
| time [s] | deviation [μs/m] | area [mm²] |
| 45 | 1.9 | 177 |
| 90 | 2.6 | 263 |
| 135 | 3.4 | 328 |

As can be seen in Table 1, the treated region area calculated by DSI increased by 48.6% for 90 s compared to 45 s, and by 85.3% for 135 s treatment, which aligns with the expected effect of HIFU treatment.

### Example 4—Calculating the Trend in Temperature Change from the Effect of Thermal Ablation on the Speed-of-Sound Shift

To calculate temperature change, slowness deviation was analyzed across six repeats of the same experimental set up as the experiments performed on ex-vivo chicken breast during thermal ablation, exemplified in Example 3 and presented in FIG. 3D including same treatment interval/time.

The slowness deviation was evaluated across multiple experiments for accurately assessing the deviation of the measured values and calculate the trend of treatment interval/time using the DSI algorithm (FIG. 4A).

In detail, in the experiments the mean temperature for each treatment interval was calculated using thermocouple (ground truth), the DSI algorithm and TSI method. The average thermocouple measurements were used as a ground-truth model to represent the expected change in temperature and facilitate the extraction of the value of the pre-calibration alpha factors. Therefore, Sound slowness and thermal strain were extracted from the DSI and TSI algorithms, respectively, and calibrated to the ground truth model. Calibration of the temperature profiles yielded values of −0.22%° C.⁻¹ for α1 and 88 ηs/m° C.⁻¹ for α2, corresponding to a change of 0.3 ms⁻¹ for a 1° C. temperature increase, in some embodiments.

These α1 and α2 values were then used for calculating the temperature prediction shown in FIG. 4B. The figure presents the temperature change assessed by a thermocouple, the TSI and the DSI methods, and indicate that all three methods detected an increase in temperature in the range of between about 3° C. and 15° C. during the 270 s of thermal ablation treatment.

For the DSI algorithm, the temperature change between 3° C. and 15° C. was measured with high thermometry precision of less than 2° C. error for temperature changes as low as 8° C., in some embodiments.

To conclude, the results presented in FIGS. 4A-4B show that the disclosed method comprising DSI algorithm provide consistent temperature assessment based on the calculated change in speed-of-sound, with low variance and a clear upward trend similar to the one expected from the temperature change during ablation therapy, e.g., heating.

### Example 5—a Trend in Temperature During HIFU after 7 Treatments of Thermal Ablation

A model of the effect of temperature change on round trip time is shown in FIG. 5A. Experiments of dense speed-of-sound shift imaging (DSI), performed according to the methods and systems of the present disclosure are presented, during HIFU in an agarose tissue-mimicking phantom, and after 7 treatments of thermal ablation, are presented in FIGS. 5B-5C and show a trend. The focal point of the HIFU ablation can be seen as it is developing in the sample.

### Example 6—DSI Exhibit Improved Performance Benchmark Relative to TSI

Finally, to emphasize the unexpected and surprising improvement in temporal processing exhibited by the DSI algorithm when utilized to calculate the change in speed-of-sound in Example 2 and Example 3, a bench test was performed to determine the average runtime of the algorithm compared to TSI. The cross-correlation parameters used in the search algorithm were chosen to optimize imaging quality as in FIG. 2A and FIGS. 3C-3D. Four random experiments were selected, and each was processed 100 times with each algorithm. The processing time for each iteration was divided by the number of frames to calculate the meantime per frame.

Advantageously, the average runtime of calculating thermal strain with the Thermal Strain Imaging TSI algorithm was 7.21±0.5 seconds/frame (equivalent to about 0.14 frames per second (rounded to about 0.15)), while average runtime of DSI for calculating the change in internal speed-of-sound was 0.33±0.004 seconds/frame (equivalent to about 3.0 frames per second).

Overall, a speedup of about ×20 or about ×21.5 in frame processing per second was achieved with DSI in calculating sound-of-speed relative to TSI in calculating thermal strain.

### Example 7—Ex-Vivo Non-Invasive Monitoring of a Change in a Temperature, During a Cryo-Ablation

An ex-vivo chicken breast sample was monitored with the DSI algorithm. Incrementally, liquid nitrogen was injected by a syringe into the sample.

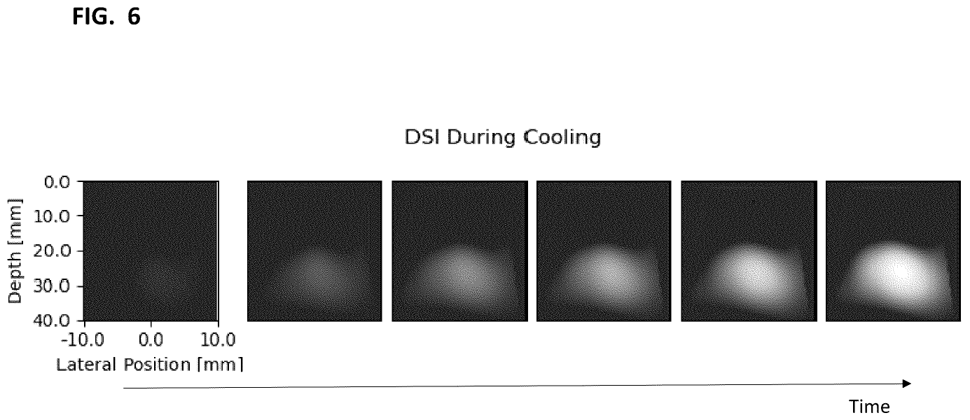

FIG. 6 shows the detected sound speed shift over time, with each frame corresponding to an injection of liquid nitrogen. The sound speed shift and change in internal tissue temperature are evaluated and quantified.

### Example 8—In-Vivo Non-Invasive Monitoring of a Change in Sound Speed, for Assessing Treatment with Contrast Agent

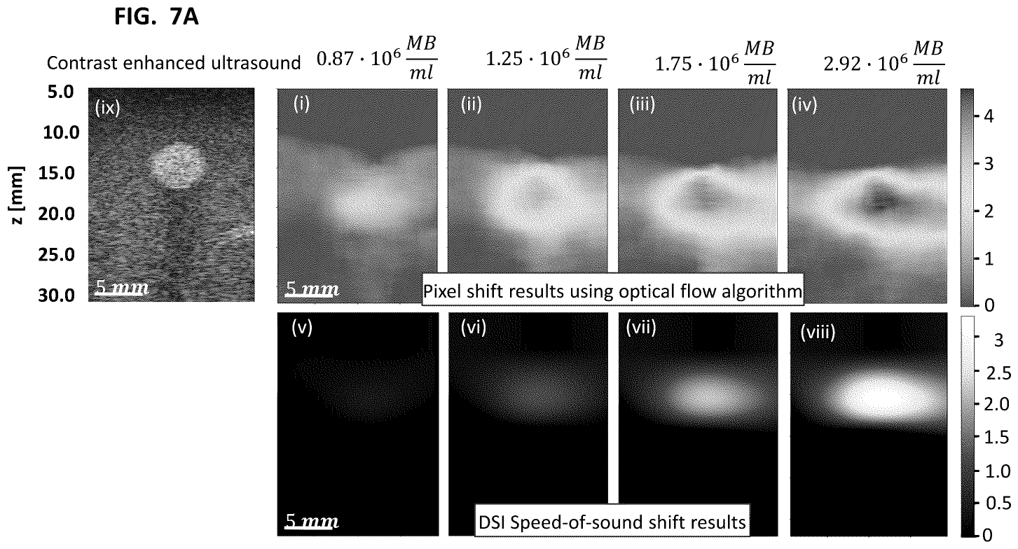

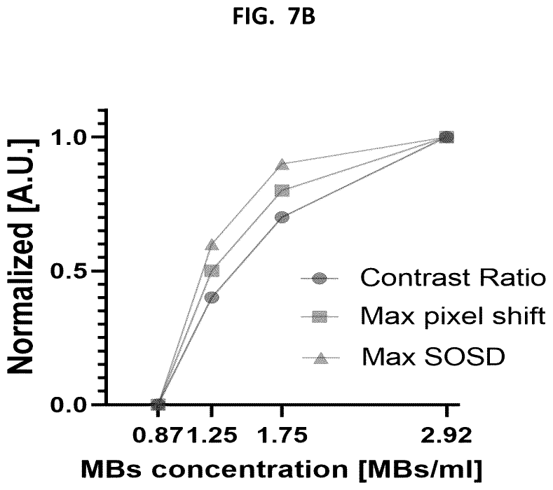

The DSI method was used for monitoring of sound-speed shift imaging wherein the shift in sound-speed was induced by microbubbles (MB) contrast agents in tissue-mimicking phantoms (FIGS. 7A-7B).

Experiments were performed using an L12-5 imaging array and a programmable ultrasound system. The performance of the DSI method (FIG. 7A (i-viii)) was compared to standard contrast enhanced imaging (FIG. 7A (ix)).

The DSI method was evaluated by imaging four increasing MB concentrations of 0.87 MB/ml, 1.25 MB/ml, 1.75 MB/ml, and 2.92×106 MB/ml as indicated in the figure. Pixel shifts were accurately detected for each of the concentrations using the optical flow algorithm (FIG. 7A (i-iv)). For each of the concentrations the speed of sound shift was calculated by solving the inverse problem to reconstruct the MB-filled inclusion (FIG. 7A (v-viii)).

Also, the results were quantified for each of the concentrations, and the normalized contrast ratios for the contrast enhanced images (blue circles), the maximal pixel shifts (red squares), and the maximal speed of sound shift (purple triangles) were calculated (FIG. 7B).

A clear relationship is shown between the MB concentration and contrast attained by the DSI algorithm.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-50. (canceled)

51. A method for non-invasive monitoring of a change in internal sound speed of a biological tissue indicative of a treatment, comprising the steps of:
(a) acquiring at least two successive ultrasound (US) images of a target area of a biological tissue, thereby obtaining raw radio frequency (RF) data of the acquired US images; and
(b) applying onto the obtained data a Dense Speed-of-Sound Shift Imaging (DSI) algorithm configured to:
(i) determine pixel shift (μm) between the at least two successive ultrasound images;
(ii) calculate a change in the internal sound speed (m/μsec) of the biological tissue based on the pixel shift; and
wherein the DSI algorithm is characterized by solving an inverse problem; and wherein the calculated change in the internal sound speed of the biological tissue comprises the solution to the inverse problem.

52. The method of claim 51, comprising estimating internal temperature change of the biological tissue based on the calculated change in the internal sound speed of the biological tissue.

53. The method of claim 51, wherein the acquisition of at least two successive ultrasound (US) images of a target area of a biological tissue is performed during a treatment performed on the tissue thereby providing indication about the treatment; and wherein the treatment can inflict a physiological change on the inherent speed-of-sound of the biological tissue and/or wherein the treatment can inflict a physiological change in the internal temperature of the biological tissue.

54. The method of claim 51, comprising a step of feeding back on the treatment; the feedback comprises providing a user with a recommendation(s) at least about terminating or continuing the treatment and/or about adjusting treatment parameters.

55. The method of claim 51, wherein the acquisition of at least two successive ultrasound (US) images comprises acquiring plane waves B-mode data.

56. The method of claim 51, wherein the acquisition of at least two successive ultrasound (US) images comprises acquiring at least nine plane waves per image.

57. The method of claim 51, wherein the calculation of the change in internal sound speed (m/μsec) comprises processing speed of at least about 0.3 frames per second.

58. The method of claim 51, wherein the calculation of the change in internal sound speed is devoid of image processing/filtering; thereby allowing increasing processing speed of the calculation time of the change in internal sound speed.

59. The method of claim 51, wherein the treatment comprises mechanical procedure, non-mechanical procedure, non-invasive procedure, or any combination thereof, or wherein the treatment comprises one or more of: high-intensity focused ultrasound (HIFU) thermal ablation therapy, cryo-ablation, adhesive bonding assessment, mechanical ablation, histotripsy, introducing a drug or other biological or chemical compound into the tissue, contrast agents, and polymer congealing, or any combination thereof; and wherein said treatment can inflict a physiological change on the inherent speed-of-sound and/or the treatment can inflict a physiological change in the internal temperature of the biological tissue.

60. The method of claim 59, wherein the thermal ablation therapy comprises contrast agents.

61. The method of claim 51, wherein the biological tissue comprises one or more tissue selected from a soft tissue, solid tissue, fat, bone, cartilage, or chemical deposit, lymphatic vessel or a blood vessel, a tumor, or any combination thereof.

62. A system for real-time non-invasive monitoring of a change in internal sound speed of a biological tissue, comprising:
(a) at least one transducer capable of acquiring at least two successive ultrasound (US) images of a target area of a biological tissue;
(b) a transmitter capable of beamforming obtained raw radio frequency (RF) data of the acquired US images;
(c) a processing unit capable of receiving the RF data and executing thereon a Dense Speed-of-Sound Shift Imaging (DSI) algorithm configured to:
(i) determine pixel shift (μm) between the at least two successive ultrasound images; and
(ii) calculate a change in the internal sound speed (m/μsec) of the biological tissue based on the pixel shift; and
(d) an output device capable of presenting the calculated change in the sound speed; and
wherein the DSI algorithm is characterized by solving an inverse problem; and wherein the calculated change in the internal sound speed of the biological tissue comprises the solution to the inverse problem comprises.

63. The system of claim 62, wherein the processing unit is configured to estimate internal temperature change of the biological tissue based on the calculated change in the internal sound speed of the biological tissue; and provide a presentation of the calculated change in temperature on the output device.

64. The system of claim 62, wherein the acquisition of at least two successive ultrasound (US) images of a target area of a biological tissue is performed during a treatment performed on the tissue thereby providing indication about the treatment; and wherein the treatment can inflict a physiological change on the inherent speed-of-sound of the biological tissue and/or wherein the treatment can inflict a physiological change in the internal temperature of the biological tissue.

65. The system of claim 62, wherein the processing unit is configured to provide feedback on the treatment; the feedback comprises providing a user with a recommendation(s) at least about terminating or continuing the treatment and/or about adjusting treatment parameters; and wherein said recommendation(s) are presented to the user by same or different output device.

66. The system of claim 62, wherein the transducer comprises a power output unit with at least one characteristic selected from: a duty cycle of about 50%, frequency of about 2 MHz, and PRF of about 20 μsec, or any combination thereof.

67. The system of claim 66, wherein the calculation of the change in internal sound speed is devoid of image processing/filtering; thereby allowing increasing processing speed of the calculation time of the change in internal sound speed.

68. A computer-implemented method for calculating a change in internal sound speed of a biological tissue indicative of a treatment, configured to:
(a) receiving raw radio frequency (RF) data of at least two successively acquired ultrasound (US) images of a target area of a biological tissue;
(b) applying onto the data a Dense Speed-of-Sound Shift Imaging (DSI) algorithm that:
(i) measures echo/pixel shift (μm) between the at least two successive ultrasound images; and
(ii) calculates a change in internal sound speed (m/μsec) of the biological tissue based on the echo/pixel shift; and
(c) providing an output comprising the calculated change in the internal sound speed of the biological tissue; and
wherein the DSI algorithm is characterized by solving an inverse problem; and wherein the calculated change in the internal sound speed of the biological tissue comprises the solution to the inverse problem comprises.

69. The computer-implemented method of claim 68, configured to estimate internal temperature change of the biological tissue based on the calculated change in the internal sound speed of the biological tissue; and provide an output comprising the calculated change in temperature.

70. The computer-implemented method of claim 68, wherein the calculation of the change in internal sound speed is devoid of image processing/filtering; thereby allowing increasing processing speed of the calculation time of the change in internal sound speed and/or
the DSI algorithm comprises a first step of echo/pixel shift calculation that is estimated with dense optic flow, and a second step of determining the change in internal sound speed that is calculated as the solution to the inverse problem.
